SEQUENCE LISTING (99 sequences)

SEQ ID NO: 1 - skipped

SEQ ID NO: 2 - protein, 18 aa, synthetic construct
  SITE 14: note = D-arginine
  SITE 11: note = D-arginine
  SITE 3: note = D-arginine
  SITE 1: note = D-arginine
  SITE 7: note = D-arginine
  SITE 9: note = D-arginine
  SITE 5: note = D-arginine
  SITE 16: note = D-arginine
RRRGRRRGRRRRGRRRRC

SEQ ID NO: 3 - protein, 17 aa, synthetic construct
  SITE 1: note = D-arginine
  SITE 5: note = D-arginine
  SITE 11: note = D-arginine
  SITE 9: note = D-arginine
  SITE 3: note = D-arginine
  SITE 16: note = D-arginine
  SITE 14: note = D-arginine
  SITE 7: note = D-arginine
RRRGRRRGRRRRGRRRR

SEQ ID NO: 4 - protein, 17 aa, synthetic construct
  SITE 1: note = D-arginine
  SITE 3: note = D-arginine
  SITE 7: note = D-arginine
  SITE 16: note = D-arginine
  SITE 14: note = D-arginine
  SITE 9: note = D-arginine
  SITE 11: note = D-arginine
  SITE 5: note = D-arginine
  MOD_RES 17: note = C-terminal carboxamide group
RRRGRRRGRRRRGRRRR

SEQ ID NO: 5 - protein, 18 aa, synthetic construct
  SITE 5: note = D-arginine
  SITE 7: note = D-arginine
  SITE 3: note = D-arginine
  SITE 1: note = D-arginine
  SITE 9: note = D-arginine
  SITE 14: note = D-arginine
  SITE 11: note = D-arginine
  SITE 4: note = 7-aminoheptanoic acid
  SITE 8: note = 5-aminovaleric acid
  SITE 13: note = 5-aminovaleric acid
  SITE 16: note = D-arginine
RRRXRRRXRRRRXRRRRC

SEQ ID NO: 6 - protein, 18 aa, synthetic construct
  SITE 2: note = D-arginine
  SITE 4: note = D-arginine
  SITE 5: note = 7-aminoheptanoic acid
  SITE 6: note = D-arginine
  SITE 8: note = D-arginine
  SITE 9: note = 5-aminovaleric acid
  SITE 10: note = D-arginine
  SITE 12: note = D-arginine
  SITE 14: note = 5-aminovaleric acid
  SITE 15: note = D-arginine
  SITE 17: note = D-arginine
CRRRXRRRXRRRRXRRRR

SEQ ID NO: 7 - protein, 17 aa, synthetic construct
  SITE 3: note = D-arginine
  SITE 4: note = bAla
  SITE 5: note = D-arginine
  SITE 7: note = D-arginine
  SITE 8: note = bAla
  SITE 9: note = D-arginine
  SITE 11: note = D-arginine
  SITE 13: note = bAla
  SITE 14: note = D-arginine
  SITE 16: note = D-arginine
  MOD_RES 17: note = C-terminal carboxamide group
  SITE 1: note = D-arginine
RRRXRRRXRRRRXRRRR

SEQ ID NO: 8 - protein, 17 aa, synthetic construct
  SITE 3: note = D-arginine
  SITE 4: note = bAla
  SITE 5: note = D-arginine
  SITE 7: note = D-arginine
  SITE 8: note = bAla
  SITE 9: note = D-arginine
  SITE 11: note = D-arginine
  SITE 13: note = bAla
  SITE 14: note = D-arginine
  SITE 16: note = D-arginine
  SITE 1: note = D-arginine
RRRXRRRXRRRRXRRRR

SEQ ID NO: 9 - protein, 17 aa, synthetic construct
  SITE 5: note = D-arginine
  SITE 7: note = D-arginine
  SITE 8: note = bAla
  SITE 9: note = D-arginine
  SITE 11: note = D-arginine
  SITE 13: note = bAla
  SITE 14: note = D-arginine
  SITE 16: note = D-arginine
  MOD_RES 17: note = C-terminal carboxamide
  SITE 4: note = bAla
RRRXRRRXRRRRXRRRR

SEQ ID NO: 10 - protein, 17 aa, synthetic construct
  SITE 1: note = D-arginine
  SITE 3: note = D-arginine
  SITE 4: note = bAla
  SITE 9: note = D-arginine
  SITE 11: note = D-arginine
  SITE 13: note = bAla
  SITE 14: note = D-arginine
  SITE 16: note = D-arginine
  MOD_RES 17: note = C-terminal carboxamide
  SITE 8: note = bAla
RRRXRRRXRRRRXRRRR

SEQ ID NO: 11 - protein, 17 aa, synthetic construct
  SITE 1: note = D-arginine
  SITE 3: note = D-arginine
  SITE 4: note = bAla
  SITE 5: note = D-arginine
  SITE 7: note = D-arginine
  SITE 8: note = bAla
  SITE 13: note = bAla
  SITE 14: note = D-arginine
  SITE 16: note = D-arginine
  MOD_RES 17: note = C-terminal carboxamide
RRRXRRRXRRRRXRRRR

SEQ ID NO: 12 - protein, 17 aa, synthetic construct
  SITE 1: note = D-arginine
  SITE 3: note = D-arginine
  SITE 4: note = bAla
  SITE 5: note = D-arginine
  SITE 7: note = D-arginine
  SITE 8: note = bAla
  SITE 9: note = D-arginine
  SITE 11: note = D-arginine
  SITE 13: note = bAla
  MOD_RES 17: note = C-terminal carboxamide
RRRXRRRXRRRRXRRRR

SEQ ID NO: 13 - protein, 17 aa, synthetic construct
  SITE 4: note = bAla
  SITE 8: note = bAla
  SITE 13: note = bAla
  MOD_RES 17: note = C-terminal carboxamide
RRRXRRRXRRRRXRRRR

SEQ ID NO: 14 - protein, 17 aa, synthetic construct
  SITE 1..3: note = D-arginine
  SITE 4: note = bAla
  SITE 5..7: note = D-arginine
  SITE 8: note = bAla
  SITE 9..12: note = D-arginine
  SITE 13: note = bAla
  SITE 14..17: note = D-arginine
  MOD_RES 17: note = C-terminal carboxamide
RRRXRRRXRRRRXRRRR

SEQ ID NO: 15 - protein, 17 aa, synthetic construct
  SITE 2: note = D-arginine
  SITE 4: note = bAla
  SITE 6: note = D-arginine
  SITE 8: note = bAla
  SITE 10: note = D-arginine
  SITE 12: note = D-arginine
  SITE 13: note = bAla
  SITE 15: note = D-arginine
  SITE 17: note = D-arginine
  MOD_RES 17: note = C-terminal carboxamide
RRRXRRRXRRRRXRRRR

SEQ ID NO: 16 - protein, 18 aa, synthetic construct
  SITE 3: note = D-arginine
  SITE 4: note = bAla
  SITE 5: note = D-arginine
  SITE 7: note = D-arginine
  SITE 8: note = bAla
  SITE 9: note = D-arginine
  SITE 11: note = D-arginine
  SITE 13: note = bAla
  SITE 14: note = D-arginine
  SITE 16: note = D-arginine
  SITE 18: note = 8-aminooctanoic acid
  SITE 1: note = D-arginine
RRRXRRRXRRRRXRRRRX

SEQ ID NO: 17 - protein, 13 aa, synthetic construct
  SITE 3: note = D-arginine
  SITE 4: note = bAla
  SITE 5: note = D-arginine
  SITE 7: note = D-arginine
  SITE 8: note = bAla
  SITE 9: note = D-arginine
  SITE 11: note = D-arginine
  SITE 13: note = bAla
  SITE 1: note = D-arginine
RRRXRRRXRRRRX

SEQ ID NO: 18 - protein, 17 aa, synthetic construct
  SITE 1: note = D-arginine
  SITE 3: note = D-arginine
  SITE 4: note = 5-aminovaleric acid
  SITE 5: note = D-arginine
  SITE 7: note = D-arginine
  SITE 8: note = 5-aminovaleric acid
  SITE 9: note = D-arginine
  SITE 11: note = D-arginine
  SITE 13: note = 5-aminovaleric acid
  SITE 14: note = D-arginine
  SITE 16: note = D-arginine
  MOD_RES 17: note = C-terminal carboxamide
RRRXRRRXRRRRXRRRR

SEQ ID NO: 19 - protein, 17 aa, synthetic construct
  SITE 3: note = D-arginine
  SITE 4: note = 5-aminovaleric acid
  SITE 5: note = D-arginine
  SITE 7: note = D-arginine
  SITE 8: note = 5-aminovaleric acid
  SITE 9: note = D-arginine
  SITE 11: note = D-arginine
  SITE 13: note = 5-aminovaleric acid
  SITE 14: note = D-arginine
  SITE 16: note = D-arginine
  SITE 1: note = D-arginine
RRRXRRRXRRRRXRRRR

SEQ ID NO: 20 - protein, 18 aa, synthetic construct
  SITE 3: note = D-arginine
  SITE 4: note = 5-aminovaleric acid
  SITE 5: note = D-arginine
  SITE 7: note = D-arginine
  SITE 8: note = 5-aminovaleric acid
  SITE 9: note = D-arginine
  SITE 11: note = D-arginine
  SITE 13: note = 5-aminovaleric acid
  SITE 14: note = D-arginine
  SITE 16: note = D-arginine
  SITE 1: note = D-arginine
RRRXRRRXRRRRXRRRRE

SEQ ID NO: 21 - protein, 17 aa, synthetic construct
  SITE 1: note = D-arginine
  SITE 3: note = D-arginine
  SITE 5: note = 5-aminovaleric acid
  SITE 6: note = D-arginine
  SITE 8: note = D-arginine
  SITE 10: note = 5-aminovaleric acid
  SITE 11: note = D-arginine
  SITE 13: note = D-arginine
  SITE 14: note = 5-aminovaleric acid
  SITE 15: note = D-arginine
  SITE 17: note = D-arginine
  MOD_RES 17: note = C-terminal carboxamide
RRRRXRRRRXRRRXRRR

SEQ ID NO: 22 - protein, 15 aa, synthetic construct
  SITE 1: note = D-arginine
  SITE 3: note = D-arginine
  SITE 4: note = 5-aminovaleric acid
  SITE 5: note = D-arginine
  SITE 7: note = D-arginine
  SITE 8: note = 5-aminovaleric acid
  SITE 9: note = D-arginine
  SITE 11: note = D-arginine
  SITE 12: note = 5-aminovaleric acid
  SITE 13: note = D-arginine
  SITE 15: note = D-arginine
  MOD_RES 15: note = C-terminal carboxamide
RRRXRRRXRRRXRRR

SEQ ID NO: 23 - protein, 13 aa, synthetic construct
  SITE 3: note = 5-aminovaleric acid
  SITE 4: note = D-arginine
  SITE 6: note = D-arginine
  SITE 7: note = 5-aminovaleric acid
  SITE 8: note = D-arginine
  SITE 10: note = D-arginine
  SITE 11: note = 5-aminovaleric acid
  SITE 12: note = D-arginine
  MOD_RES 13: note = C-terminal carboxamide
  SITE 1: note = D-arginine
RRXRRRXRRRXRR

SEQ ID NO: 24 - protein, 13 aa, synthetic construct
  SITE 1: note = D-arginine
  SITE 3: note = D-arginine
  SITE 4: note = 5-aminovaleric acid
  SITE 5: note = D-arginine
  SITE 7: note = 5-aminovaleric acid
  SITE 8: note = D-arginine
  SITE 10: note = D-arginine
  SITE 11: note = 5-aminovaleric acid
  SITE 12: note = D-arginine
  MOD_RES 13: note = C-terminal carboxamide
RRRXRRXRRRXRR

SEQ ID NO: 25 - protein, 13 aa, synthetic construct
  SITE 1: note = D-arginine
  SITE 3: note = 5-aminovaleric acid
  SITE 4: note = D-arginine
  SITE 6: note = D-arginine
  SITE 7: note = 5-aminovaleric acid
  SITE 8: note = D-arginine
  SITE 10: note = 5-aminovaleric acid
  SITE 11: note = D-arginine
  SITE 13: note = D-arginine
  MOD_RES 13: note = C-terminal carboxamide
RRXRRRXRRXRRR

SEQ ID NO: 26 - protein, 13 aa, synthetic construct
  SITE 1: note = D-arginine
  SITE 3: note = 5-aminovaleric acid
  SITE 4: note = D-arginine
  SITE 7: note = D-arginine
  SITE 9: note = D-arginine
  SITE 10: note = 5-aminovaleic acid
  SITE 11: note = D-arginine
  SITE 13: note = D-arginine
  MOD_RES 13: note = C-terminal carboxamide
RRXRRXRRRXRRR

SEQ ID NO: 27 - protein, 13 aa, synthetic construct
  SITE 1: note = D-arginine
  SITE 3: note = D-arginine
  SITE 4: note = 5-aminovaleric acid
  SITE 5: note = D-arginine
  SITE 7: note = D-arginine
  SITE 8: note = 5-aminovaleric acid
  SITE 9: note = D-arginine
  SITE 11: note = 5-aminovaleric acid
  SITE 12: note = D-arginine
  MOD_RES 13: note = C-terminal carboxamide
RRRXRRRXRRXRR

SEQ ID NO: 28 - protein, 13 aa, synthetic construct
  SITE 1: note = D-arginine
  SITE 3: note = D-arginine
  SITE 4: note = 5-aminovaleric acid
  SITE 5: note = D-arginine
  SITE 7: note = 5-aminovaleric acid
  SITE 8: note = D-arginine
  SITE 10: note = 5-aminovaleric acid
  SITE 11: note = D-arginine
  SITE 13: note = D-arginine
  MOD_RES 13: note = C-terminal carboxamide
RRRXRRXRRXRRR

SEQ ID NO: 29 - protein, 17 aa, synthetic construct
  SITE 1: note = D-arginine
  SITE 3: note = D-arginine
  SITE 4: note = 8-aminooctanoic acid
  SITE 5: note = D-arginine
  SITE 7: note = D-arginine
  SITE 8: note = 8-aminooctanoic acid
  SITE 9: note = D-arginine
  SITE 11: note = D-arginine
  SITE 13: note = 8-aminooctanoic acid
  SITE 14: note = D-arginine
  SITE 16: note = D-arginine
  MOD_RES 17: note = C-terminal carboxamide
RRRXRRRXRRRRXRRRR

SEQ ID NO: 30 - protein, 17 aa, synthetic construct
  SITE 1: note = D-arginine
  SITE 3: note = D-arginine
  SITE 4: note = 8-aminooctanoic acid
  SITE 5: note = D-arginine
  SITE 7: note = D-arginine
  SITE 8: note = 8-aminooctanoic acid
  SITE 9: note = D-arginine
  SITE 11: note = D-arginine
  SITE 13: note = 8-aminooctanoic acid
  SITE 14: note = D-arginine
  SITE 16: note = D-arginine
RRRXRRRXRRRRXRRRR

SEQ ID NO: 31 - protein, 17 aa, synthetic construct
  MOD_RES 1: note = N-terminal acetylation
  SITE 1: note = D-arginine
  SITE 3: note = D-arginine
  SITE 4: note = 8-aminooctanoic acid
  SITE 5: note = D-arginine
  SITE 7: note = D-arginine
  SITE 8: note = 8-aminooctanoic acid
  SITE 9: note = D-arginine
  SITE 11: note = D-arginine
  SITE 13: note = 8-aminooctanoic acid
  SITE 14: note = D-arginine
  SITE 16: note = D-arginine
  MOD_RES 17: note = C-terminal carboxamide
RRRXRRRXRRRRXRRRR

SEQ ID NO: 32 - protein, 17 aa, synthetic construct
  SITE 1: note = D-arginine
  SITE 3: note = D-arginine
  SITE 4: note = 10-aminodecanoic acid
  SITE 5: note = D-arginine
  SITE 7: note = D-arginine
  SITE 8: note = 8-aminooctanoic acid
  SITE 9: note = D-arginine
  SITE 11: note = D-arginine
  SITE 13: note = 8-aminooctanoic acid
  SITE 14: note = D-arginine
  SITE 16: note = D-arginine
RRRXRRRXRRRRXRRRR

SEQ ID NO: 33 - protein, 17 aa, synthetic construct
  SITE 1: note = D-arginine
  SITE 3: note = D-arginine
  SITE 4: note = 10-aminodecanoic acid
  SITE 5: note = D-arginine
  SITE 7: note = D-arginine
  SITE 8: note = 8-aminooctanoic acid
  SITE 9: note = D-arginine
  SITE 11: note = D-arginine
  SITE 13: note = 8-aminooctanoic acid
  SITE 14: note = D-arginine
  SITE 16: note = D-arginine
  MOD_RES 17: note = C-terminal carboxamide
RRRXRRRXRRRRXRRRR

SEQ ID NO: 34 - protein, 18 aa, synthetic construct
  SITE 1: note = D-arginine
  SITE 3: note = D-arginine
  SITE 4: note = 10-aminodecanoic acid
  SITE 5: note = D-arginine
  SITE 7: note = D-arginine
  SITE 8: note = 8-aminooctanoic acid
  SITE 9: note = D-arginine
  SITE 11: note = D-arginine
  SITE 13: note = 8-aminooctanoic acid
  SITE 14: note = D-arginine
  SITE 16: note = D-arginine
  SITE 18: note = 8-aminooctanoic acid
RRRXRRRXRRRRXRRRRX

SEQ ID NO: 35 - protein, 17 aa, synthetic construct
  SITE 1: note = D-arginine
  SITE 3: note = D-arginine
  SITE 4: note = N-butylglycine
  SITE 5: note = D-arginine
  SITE 7: note = D-arginine
  SITE 8: note = N-butylglycine
  SITE 9: note = D-arginine
  SITE 11: note = D-arginine
  SITE 13: note = N-butylglycine
  SITE 14: note = D-arginine
  SITE 16: note = D-arginine
  MOD_RES 17: note = C-terminal carboxamide
RRRXRRRXRRRRXRRRR

SEQ ID NO: 36 - protein, 13 aa, synthetic construct
  SITE 1: note = D-arginine
  SITE 3: note = N-butylglycine
  SITE 4: note = D-arginine
  SITE 6: note = N-butylglycine
  SITE 7: note = D-arginine
  SITE 9: note = D-arginine
  SITE 10: note = N-butylglycine
  SITE 11: note = D-arginine
  SITE 13: note = D-arginine
  MOD_RES 13: note = C-terminal carboxamide
RRXRRXRRRXRRR

SEQ ID NO: 37 - protein, 17 aa, synthetic construct
  SITE 1: note = D-arginine
  SITE 3: note = D-arginine
  SITE 4: note = L-alpha-methylleucine
  SITE 8: note = L-alpha-methylleucine
  SITE 13: note = L-alpha-methylleucine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
  MOD_RES 17: note = C-terminal carboxamide
RRRXRRRXRRRRXRRRR

SEQ ID NO: 38 - protein, 17 aa, synthetic construct
  SITE 4: note = N-(3-aminopropyl)glycine
  SITE 8: note = N-(3-aminopropyl)glycine
  SITE 13: note = N-(3-aminopropyl)glycine
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
  MOD_RES 17: note = C-terminal carboxamide
RRRXRRRXRRRRXRRRR

SEQ ID NO: 39 - protein, 13 aa, synthetic construct
  SITE 3: note = N-(3-aminopropyl)glycine
  SITE 6: note = N-(3-aminopropyl)glycine
  SITE 10: note = N-(3-aminopropyl)glycine
  SITE 1: note = D-arginine
  SITE 4: note = D-arginine
  SITE 7: note = D-arginine
  SITE 9: note = D-arginine
  SITE 11: note = D-arginine
  SITE 13: note = D-arginine
  MOD_RES 13: note = C-terminal carboxamide
RRXRRXRRRXRRR

SEQ ID NO: 40 - protein, 17 aa, synthetic construct
  SITE 4: note = 2-amino-3-guanidino-propionic acid
  SITE 8: note = 2-amino-3-guanidino-propionic acid
  SITE 13: note = 2-amino-3-guanidino-propionic acid
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
  MOD_RES 17: note = C-terminal carboxamide
RRRXRRRXRRRRXRRRR

SEQ ID NO: 41 - protein, 17 aa, synthetic construct
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
  MOD_RES 17: note = C-terminal carboxamide
  SITE 4: note = N-(2-aminoethyl)glycine
  SITE 8: note = 5-aminovaleric acid
  SITE 13: note = 5-aminovaleric acid
RRRXRRRXRRRRXRRRR

SEQ ID NO: 42 - protein, 17 aa, synthetic construct
  SITE 4: note = 5-aminovaleric acid
  SITE 13: note = 5-aminovaleric acid
  SITE 8: note = N-(2-aminoethyl)glycine
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 43 - protein, 17 aa, synthetic construct
  SITE 4: note = 5-aminovaleric acid
  SITE 8: note = 5-aminovaleric acid
  SITE 13: note = N-(2-aminoethyl)glycine
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 44 - protein, 17 aa, synthetic construct
  SITE 4: note = N-(2-aminoethyl)glycine
  SITE 8: note = N-(2-aminoethyl)glycine
  SITE 13: note = 5-aminovaleric acid
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 45 - protein, 17 aa, synthetic construct
  SITE 4: note = 5-aminovaleric acid
  SITE 8: note = N-(2-aminoethyl)glycine
  SITE 13: note = N-(2-aminoethyl)glycine
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 46 - protein, 17 aa, synthetic construct
  SITE 4: note = N-(2-aminoethyl)glycine
  SITE 8: note = N-(2-aminoethyl)glycine
  SITE 13: note = N-(2-aminoethyl)glycine
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 47 - protein, 17 aa, synthetic construct
  SITE 4: note = 5-amino-3-oxapentanoic acid
  SITE 8: note = 5-aminovaleric acid
  SITE 13: note = 5-aminovaleric acid
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 48 - protein, 17 aa, synthetic construct
  SITE 4: note = 5-aminovaleric acid
  SITE 13: note = 5-aminovaleric acid
  SITE 8: note = 5-amino-3-oxapentanoic acid
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 49 - protein, 17 aa, synthetic construct
  SITE 4: note = 5-aminovaleric acid
  SITE 8: note = 5-aminovaleric acid
  SITE 13: note = 5-amino-3-oxapentanoic acid
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 50 - protein, 17 aa, synthetic construct
  SITE 4: note = 5-amino-3-oxapentanoic acid
  SITE 8: note = 5-amino-3-oxapentanoic acid
  SITE 13: note = 5-aminovaleric acid
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 51 - protein, 17 aa, synthetic construct
  SITE 4: note = 5-aminovaleric acid
  SITE 8: note = 5-amino-3-oxapentanoic acid
  SITE 13: note = 5-amino-3-oxapentanoic acid
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 14: note = D-arginine
  SITE 11: note = D-arginine
  SITE 16: note = D-arginine
RRRXRRRXRRRRXRRRR

SEQ ID NO: 52 - protein, 17 aa, synthetic construct
  SITE 4: note = 5-amino-3-oxapentanoic acid
  SITE 8: note = 5-amino-3-oxapentanoic acid
  SITE 13: note = 5-amino-3-oxapentanoic acid
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 53 - protein, 17 aa, synthetic construct
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRPRRRPRRRRPRRRR

SEQ ID NO: 54 - protein, 17 aa, synthetic construct
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRPRRRPRRRRPRRRR

SEQ ID NO: 55 - protein, 17 aa, synthetic construct
  MOD_RES 1: note = N-terminal acetylation
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRPRRRPRRRRPRRRR

SEQ ID NO: 56 - protein, 17 aa, synthetic construct
  MOD_RES 1: note = N-terminal methane sulfonamide
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRPRRRPRRRRPRRRR

SEQ ID NO: 57 - protein, 17 aa, synthetic construct
  MOD_RES 1: note = N-terminal 4-fluorophenyl-isocyanate
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRPRRRPRRRRPRRRR

SEQ ID NO: 58 - protein, 17 aa, synthetic construct
  MOD_RES 1: note = N-terminal cyclohexyl-isocyanate
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRPRRRPRRRRPRRRR

SEQ ID NO: 59 - protein, 17 aa, synthetic construct
  MOD_RES 1: note = N-terminal morpholineacetic acid
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRPRRRPRRRRPRRRR

SEQ ID NO: 60 - protein, 17 aa, synthetic construct
  MOD_RES 17: note = C-terminal carboxamide
  SITE 1: note = D-arginine
  SITE 3: note = D-arginine
  SITE 6: note = D-arginine
  SITE 8: note = D-arginine
  SITE 11: note = D-arginine
  SITE 13: note = D-arginine
  SITE 15: note = D-arginine
  SITE 17: note = D-arginine
RRRRPRRRRPRRRPRRR

SEQ ID NO: 61 - protein, 16 aa, synthetic construct
  MOD_RES 16: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 13: note = D-arginine
  SITE 15: note = D-arginine
RRRRRPRRRRRPRRRR

SEQ ID NO: 62 - protein, 15 aa, synthetic construct
  MOD_RES 15: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 13: note = D-arginine
  SITE 15: note = D-arginine
RRRRRRRPRRRRRRR

SEQ ID NO: 63 - protein, 18 aa, synthetic construct
  MOD_RES 18: note = C-terminal carboxamide
  SITE 2: note = D-arginine
  SITE 4: note = D-arginine
  SITE 6: note = D-arginine
  SITE 8: note = D-arginine
  SITE 10: note = D-arginine
  SITE 12: note = D-arginine
  SITE 15: note = D-arginine
  SITE 17: note = D-arginine
RRRRPRRRPRRRRPRRRR

SEQ ID NO: 64 - protein, 19 aa, synthetic construct
  MOD_RES 19: note = C-terminal carboxamide
  SITE 2: note = D-arginine
  SITE 4: note = D-arginine
  SITE 6: note = D-arginine
  SITE 8: note = D-arginine
  SITE 11: note = D-arginine
  SITE 13: note = D-arginine
  SITE 16: note = D-arginine
  SITE 18: note = D-arginine
RRRRPRRRRPRRRRPRRRR

SEQ ID NO: 65 - protein, 16 aa, synthetic construct
  MOD_RES 16: note = C-terminal carboxamide
  SITE 2: note = D-arginine
  SITE 4: note = D-arginine
  SITE 6: note = D-arginine
  SITE 8: note = D-arginine
  SITE 11: note = D-arginine
  SITE 14: note = D-arginine
  SITE 16: note = D-arginine
RRRRPRRRPRRRPRRR

SEQ ID NO: 66 - protein, 15 aa, synthetic construct
  MOD_RES 15: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 13: note = D-arginine
  SITE 15: note = D-arginine
RRRPRRRPRRRPRRR

SEQ ID NO: 67 - protein, 14 aa, synthetic construct
  MOD_RES 14: note = C-terminal carboxamide
  SITE 1: note = D-arginine
  SITE 3: note = D-arginine
  SITE 6: note = D-arginine
  SITE 8: note = D-arginine
  SITE 11: note = D-arginine
  SITE 13: note = D-arginine
RRRRPRRRRPRRRR

SEQ ID NO: 68 - protein, 13 aa, synthetic construct
  MOD_RES 13: note = C-terminal carboxamide
  SITE 1: note = D-arginine
  SITE 3: note = D-arginine
  SITE 5: note = D-arginine
  SITE 8: note = D-arginine
  SITE 10: note = D-arginine
  SITE 12: note = D-arginine
RRRPRRPRRRPRR

SEQ ID NO: 69 - protein, 13 aa, synthetic construct
  MOD_RES 13: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 12: note = D-arginine
RRRPRRRPRRPRR

SEQ ID NO: 70 - protein, 13 aa, synthetic construct
  SITE 1: note = D-arginine
  SITE 3: note = D-arginine
  SITE 5: note = D-arginine
  SITE 8: note = D-arginine
  SITE 11: note = D-arginine
  SITE 13: note = D-arginine
  MOD_RES 13: note = C-terminal carboxamide
RRRPRRPRRPRRR

SEQ ID NO: 71 - protein, 13 aa, synthetic construct
  MOD_RES 13: note = C-terminal carboxamide
  SITE 1: note = D-arginine
  SITE 4: note = D-arginine
  SITE 6: note = D-arginine
  SITE 8: note = D-arginine
  SITE 10: note = D-arginine
  SITE 12: note = D-arginine
RRPRRRPRRRPRR

SEQ ID NO: 72 - protein, 13 aa, synthetic construct
  SITE 1: note = D-arginine
  SITE 4: note = D-arginine
  SITE 6: note = D-arginine
  SITE 8: note = D-arginine
  SITE 11: note = D-arginine
  SITE 13: note = D-arginine
  MOD_RES 13: note = C-terminal carboxamide
RRPRRRPRRPRRR

SEQ ID NO: 73 - protein, 13 aa, synthetic construct
  MOD_RES 13: note = C-terminal carboxamide
  SITE 1: note = D-arginine
  SITE 4: note = D-arginine
  SITE 7: note = D-arginine
  SITE 9: note = D-arginine
  SITE 11: note = D-arginine
  SITE 13: note = D-arginine
RRPRRPRRRPRRR

SEQ ID NO: 74 - protein, 17 aa, synthetic construct
  SITE 4: note = isonipecotic acid
  SITE 8: note = isonipecotic acid
  SITE 13: note = isonipecotic acid
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 75 - protein, 17 aa, synthetic construct
  MOD_RES 17: note = C-terminal carboxamide
  SITE 4: note = L-2-indanylglycine
  SITE 8: note = L-2-indanylglycine
  SITE 13: note = L-2-indanylglycine
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 76 - protein, 17 aa, synthetic construct
  MOD_RES 17: note = C-terminal carboxamide
  SITE 4: note = 1,2,3,4-tetrahydroisoquinoline-3-carboxylic acid
  SITE 8: note = 1,2,3,4-tetrahydroisoquinoline-3-carboxylic acid
  SITE 13: note = 1,2,3,4-tetrahydroisoquinoline-3-carboxylic acid
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 77 - protein, 17 aa, synthetic construct
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
  SITE 4: note = (4'-pyridyl)alanine
  SITE 8: note = (4'-pyridyl)alanine
  SITE 13: note = (4'-pyridyl)alanine
  MOD_RES 17: note = C-terminal carboxamide
RRRXRRRXRRRRXRRRR

SEQ ID NO: 78 - protein, 13 aa, synthetic construct
  MOD_RES 13: note = C-terminal carboxamide
  SITE 3: note = (4'-pyridyl)alanine
  SITE 6: note = (4'-pyridyl)alanine
  SITE 10: note = (4'-pyridyl)alanine
  SITE 1: note = D-arginine
  SITE 4: note = D-arginine
  SITE 7: note = D-arginine
  SITE 9: note = D-arginine
  SITE 11: note = D-arginine
  SITE 13: note = D-arginine
RRXRRXRRRXRRR

SEQ ID NO: 79 - protein, 13 aa, synthetic construct
  MOD_RES 13: note = C-terminal carboxamide
  SITE 3: note = 2-amino-3-guanidino-propionic acid
  SITE 6: note = 2-amino-3-guanidino-propionic acid
  SITE 10: note = 2-amino-3-guanidino-propionic acid
  SITE 1: note = D-arginine
  SITE 4: note = D-arginine
  SITE 7: note = D-arginine
  SITE 9: note = D-arginine
  SITE 11: note = D-arginine
  SITE 13: note = D-arginine
RRXRRXRRRXRRR

SEQ ID NO: 80 - protein, 17 aa, synthetic construct
  SITE 4: note = N-(2-aminoethyl)glycine
  SITE 8: note = N-(2-aminoethyl)glycine
  SITE 13: note = N-(2-aminoethyl)glycine
  MOD_RES 17: note = C-terminal carboxamide
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 81 - protein, 17 aa, synthetic construct
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
  MOD_RES 17: note = C-terminal carboxamide
  SITE 4: note = N-(4-piperidinyl)glycine
  SITE 8: note = N-(4-piperidinyl)glycine
  SITE 13: note = N-(4-piperidinyl)glycine
RRRXRRRXRRRRXRRRR

SEQ ID NO: 82 - protein, 17 aa, synthetic construct
  MOD_RES 17: note = C-terminal carboxamide
  SITE 4: note = (S)-N-(1-phenylethyl)glycine
  SITE 8: note = (S)-N-(1-phenylethyl)glycine
  SITE 13: note = (S)-N-(1-phenylethyl)glycine
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 83 - protein, 17 aa, synthetic construct
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
  MOD_RES 17: note = C-terminal carboxamide
  SITE 4: note = N-benzylglycine
  SITE 8: note = N-benzylglycine
  SITE 13: note = N-benzylglycine
RRRXRRRXRRRRXRRRR

SEQ ID NO: 84 - protein, 13 aa, synthetic construct
  SITE 3: note = N-(2-aminoethyl)glycine
  SITE 6: note = N-(2-aminoethyl)glycine
  SITE 10: note = N-(2-aminoethyl)glycine
  MOD_RES 13: note = C-terminal carboxamide
  SITE 1: note = D-arginine
  SITE 4: note = D-arginine
  SITE 7: note = D-arginine
  SITE 9: note = D-arginine
  SITE 11: note = D-arginine
  SITE 13: note = D-arginine
RRXRRXRRRXRRR

SEQ ID NO: 85 - protein, 13 aa, synthetic construct
  SITE 3: note = N-(4-piperidinyl)glycine
  SITE 6: note = N-(4-piperidinyl)glycine
  SITE 10: note = N-(4-piperidinyl)glycine
  MOD_RES 13: note = C-terminal carboxamide
  SITE 1: note = D-arginine
  SITE 4: note = D-arginine
  SITE 7: note = D-arginine
  SITE 9: note = D-arginine
  SITE 11: note = D-arginine
  SITE 13: note = D-arginine
RRXRRXRRRXRRR

SEQ ID NO: 86 - protein, 13 aa, synthetic construct
  MOD_RES 13: note = C-terminal carboxamide
  SITE 3: note = (S)-N-(1-phenylethyl)glycine
  SITE 6: note = (S)-N-(1-phenylethyl)glycine
  SITE 10: note = (S)-N-(1-phenylethyl)glycine
  SITE 1: note = D-arginine
  SITE 4: note = D-arginine
  SITE 7: note = D-arginine
  SITE 9: note = D-arginine
  SITE 11: note = D-arginine
  SITE 13: note = D-arginine
RRXRRXRRRXRRR

SEQ ID NO: 87 - protein, 13 aa, synthetic construct
  MOD_RES 13: note = C-terminal carboxamide
  SITE 3: note = N-benzylglycine
  SITE 6: note = N-benzylglycine
  SITE 10: note = N-benzylglycine
  SITE 1: note = D-arginine
  SITE 4: note = D-arginine
  SITE 7: note = D-arginine
  SITE 9: note = D-arginine
  SITE 11: note = D-arginine
  SITE 13: note = D-arginine
RRXRRXRRRXRRR

SEQ ID NO: 88 - protein, 17 aa, synthetic construct
  MOD_RES 17: note = C-terminal carboxamide
  SITE 2: note = homoarginine
  SITE 6: note = homoarginine
  SITE 10: note = homoarginine
  SITE 12: note = homoarginine
  SITE 15: note = homoarginine
  SITE 17: note = homoarginine
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RXRPRXRPRXRXPRXRX

SEQ ID NO: 89 - protein, 17 aa, synthetic construct
  MOD_RES 17: note = C-terminal carboxamide
  SITE 4: note = 5-aminovaleric acid
  SITE 8: note = 5-aminovaleric acid
  SITE 13: note = 5-aminovaleric acid
  SITE 2: note = homoarginine
  SITE 6: note = homoarginine
  SITE 10: note = homoarginine
  SITE 12: note = homoarginine
  SITE 15: note = homoarginine
  SITE 17: note = homoarginine
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RXRXRXRXRXRXXRXRX

SEQ ID NO: 90-91 - skipped (2 entries)

SEQ ID NO: 92 - protein, 17 aa, synthetic construct
  MOD_RES 17: note = C-terminal carboxamide
  SITE 4: note = 12-aminododecanoic acid
  SITE 8: note = 12-aminododecanoic acid
  SITE 13: note = 12-aminododecanoic acid
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRXRRRXRRRRXRRRR

SEQ ID NO: 93 - protein, 17 aa, synthetic construct
  MOD_RES 17: note = C-terminal carboxamide
  SITE 17: note = homoleucine
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RRRPRRRPRRRRPRRRX

SEQ ID NO: 94 - protein, 17 aa, synthetic construct
  SITE 1: note = homoleucine
  MOD_RES 17: note = C-terminal carboxamide
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
XRRPRRRPRRRRPRRRR

SEQ ID NO: 95 - protein, 17 aa, synthetic construct
  SITE 1: note = homoleucine
  SITE 17: note = homoleucine
  MOD_RES 17: note = C-terminal carboxamide
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
XRRPRRRPRRRRPRRRX

SEQ ID NO: 96 - protein, 17 aa, synthetic construct
  MOD_RES 17: note = C-terminal carboxamide
  SITE 4: note = 8-aminooctanoic acid
  SITE 8: note = 8-aminooctanoic acid
  SITE 13: note = 8-aminooctanoic acid
  SITE 2: note = homoarginine
  SITE 6: note = homoarginine
  SITE 10: note = homoarginine
  SITE 12: note = homoarginine
  SITE 15: note = homoarginine
  SITE 17: note = homoarginine
  SITE 1: note = D-arginine
  SITE 3: note = D-arginine
  SITE 5: note = D-arginine
  SITE 7: note = D-arginine
  SITE 9: note = D-arginine
  SITE 11: note = D-arginine
  SITE 14: note = D-arginine
  SITE 16: note = D-arginine
RXRXRXRXRXRXXRXRX

SEQ ID NO: 97 - protein, 16 aa, synthetic construct
  MOD_RES 16: note = C-terminal carboxamide
  SITE 4: note = 8-aminooctanoic acid
  SITE 8: note = 8-aminooctanoic acid
  SITE 12: note = 8-aminooctanoic acid
  SITE 1: note = homoarginine
  SITE 3: note = homoarginine
  SITE 5: note = homoarginine
  SITE 7: note = homoarginine
  SITE 9: note = homoarginine
  SITE 11: note = homoarginine
  SITE 13: note = homoarginine
  SITE 15: note = homoarginine
XRXXXRXXXRXXXRXR

SEQ ID NO: 98 - protein, 16 aa, synthetic construct
  MOD_RES 16: note = C-terminal carboxamide
  SITE 1: note = homoarginine
  SITE 3: note = homoarginine
  SITE 5: note = homoarginine
  SITE 7: note = homoarginine
  SITE 9: note = homoarginine
  SITE 11: note = homoarginine
  SITE 13: note = homoarginine
  SITE 15: note = homoarginine
XRXPXRXPXRXPXRXR

SEQ ID NO: 99 - protein, 12 aa, synthetic construct
GRKKRRQRRRPQ

(19) United States
(12) Patent Application Publication
(10) Pub. No.: US 2024/0360177 A1
(43) Pub. Date: Oct. 31, 2024

(54) PEPTIDES WITH ANTIMICROBIAL ACTIVITIES

(71) Applicant: AimMax Therapeutics, Inc., Durham, NC (US)

(72) Inventors: Laurene Wang, Chapel Hill, NC (US); David E. Pereira, Salem, VA (US); Dale J. . Christensen, Cary, NC (US); Kara S. Keedy, Durham, NC (US); Gregory J. Pacofsky, Raleigh, NC (US); Derek J. Nunez, Raleigh, NC (US)

(21) Appl. No.: 18/687,807

(22) PCT Filed: Sep. 1, 2022
(86) PCT No.: PCT/US2022/042312

§ 371 Date: Feb. 28, 2024

Related U.S. Application Data
(60) Provisional application No. 63/268,158, filed on Feb. 17, 2022.
(60) Provisional application No. 63/260,836, filed on Sep. 1, 2021.

(51) Int. Cl. C07K 7/08; A61K 38/00; A61P 31/04; A61P 31/10; C07K 7/06

(52) U.S. Cl. CPC C07K 7/08; A61P 31/04; A61P 31/10; C07K 7/06; A61K 38/00

(57) ABSTRACT

The disclosure includes peptides of Formula (I): S1-[block-1]ₘ-x-[block-2]ₙ-y-[block-3]ₒ-z-[block-4]ₚ-S2. Also included are pharmaceutical compositions containing the peptides and methods of treating microbial infections using the peptides.

### FIELD OF THE INVENTION

The present invention relates to novel arginine-containing peptides (ACP) as treatment for fungal infections. The novel peptides comprise 2 to 4 “blocks” each of which comprises 2 to 7 L-arginine and/or D-arginine and/or homoarginine amino acids connected by a “linker” comprising a single amino acid or amine acid between any 2 “blocks” of arginines and/or homoarginines. These peptides may also include modifications at the N- and/or C-terminus.

### BACKGROUND OF THE INVENTION

Invasive candidiasis is a significant cause of morbidity and mortality in the US, with associated high mortality rates, despite appropriate antifungal therapy (Pfaller M A et al., Clin Microbiol Rev 2007, 20(1): p. 133-63). Candida albicans is the most common cause, followed by C. glabrata, and together these two pathogens account for nearly 70% of all candidemias. C. parapsilosis and C. tropicalis are responsible for the majority of the remaining cases, with various other species accounting for ≤3% of infections (Lockhart S R et al., J Clin Microbiol 2012, 50(11): p. 3435-42; Diagn Microbiol Infect Dis 2012, 74(4): p. 323-31; CDC, “Antibiotic Resistance Threats in the United States” CDC: Atlanta, G A. 2019, p. 1-138). There are three classes of antifungal drugs available for the treatment of invasive candidiasis: polyenes (e.g., amphotericin B), azoles, and echinocandins. Each of these classes has limitations, including a narrow therapeutic window with some amphotericin products, drug-drug interactions and large variability in pharmacokinetics (PK) with azoles, lack of concentrations in the brain and urine with echinocandins, as well as high and/or increasing rates of antifungal resistance in various Candida species (Pappas P G et al., Clin Infect Dis. 2016, 62(4): p. e1-50; Ashley E S D et al. Pharmacology of Systemic Antifungal Agents, Clinical Infectious Diseases 43(Supplement 1) 2006: p. S28-S39; Wiederhold N P, Infect Drug Resist10(doi) 2017: p. 249-259. PMC5587015; Bidaud A L et al., J Mycol Med 2018, 28(3): p. 568-573).

Approximately 7% of candidemias are resistant to at least one class of antifungals (CDC, “Antibiotic Resistance Threats in the United States” CDC: Atlanta, G A. 2019, p. 1-138). C. glabrata is of particular concern due to the severity of disease and rising rates of echinocandin resistance on a background of high azole-resistance (Vallabhaneni S et al., Open Forum Infect Dis 2015, 2(4): p. ofv163. PMC4677623). Although the overall prevalence is low, C. krusei also makes up a large percentage of these resistant candidemias as this species is intrinsically resistant to fluconazole (Lockhart S R et al., J Clin Microbiol 2012, 50(11): p. 3435-42). C. auris is another rare but concerning pathogen that has rapidly spread around the world since it first emerged in 2009 with high rates of resistance (90% resistant to 1 class, 30% resistant to 2, and some resistant to all available antifungals) (Forsberg K K et al., Med Mycol. 2019, 57(1): p. 1-12).

Antimicrobial peptides (AMP) enriched in cationic amino acids have been noted for positive attributes of rapid cidality (frequently through interactions with the negatively charged microbial membranes leading to disruption), low propensity for developing resistance, and low potential for off-target effects or drug-drug interactions (Hancock R E et al., Nat Biotechnol. 2006, 24(12): p. 1551-7. doi: 10.1038/nbt1267; Gordon Y J et al., Curr Eye Res. 2005, 30(7): p. 505-15; Lau J L et al., Bioorg Med Chem 2018, 26(10): p. 2700-2707; Lewies A et al., Probiotics Antimicrob Proteins 2019, 11(2): p. 370-381). Due to structural differences and unique mechanisms, they are less likely to suffer from cross-resistance with traditional antimicrobials. Success in the clinical development of AMPs historically has largely been limited by toxicities due to host cell membrane disruption (e.g., hemolysis and cytotoxicity), reduced or loss of activity under physiological conditions, and/or rapid enzymatic degradation in vivo (Koo H B et al., Peptide Science 2019, 111(5): p. e24122; Mahlapuu M et al., Frontiers in cellular and infection microbiology 2016, 6: p. 194-194).

The need for novel new antimicrobial therapeutics, in particular new antifungal therapeutics, that are safe and effective and can overcome fungal resistance remains a major unmet medical need. The compounds of the present invention are directed to meeting this unmet medical need, in particular the need for new therapeutics to treat fungal infections. The compounds of the present invention have potent antifungal activity, tolerability, selectivity, and stability.

### SUMMARY OF INVENTION

The present invention concerns arginine-containing peptides (ACP) for use in treating microbial infections, in particular fungal infections.

In one aspect, the present invention provides for peptides having the structure of Formula I:

S1-[block-1]ₘ-x-[block-2]ₙ-y-[block-3]ₒ-z-[block-4]ₚ-S2 Formula I
SEQ ID NO: 1
or a pharmaceutically acceptable salt thereof, wherein
m, n, o and p independently are 0 or 1, with 0 representing absent and 1 representing present, wherein at least two of m, n, o and p are 1;
block-1, block-2, block-3, and block-4 independently comprise 2 to 7 amino acids each independently selected from an L-arginine (R), D-arginine (r) and homoarginine (Har);
S1 and S2 are each independently an amino acid or amine acid other than an R, r or Har, and are independently present or absent;
x, y, and z are each a linker, and each linker is, independently, present or absent and comprised of a single amino acid or amine acid selected from:
proline (P), glycine (G), 3-aminopropionic acid (β-alanine, Apr), 4-aminobutyric acid (Aba), 5-aminovaleric acid (Ava), 6-aminohexanoic acid (Ahx), 7-aminoheptanoic acid (Ahp), 8-aminooctanoic acid (Aoa), 9-aminononanoic acid (Ana), 10-aminodecanoic acid (Ada), 11-aminoundecanoic acid (Aun), 12-aminododecanoic acid (Ado), 13-aminotridecanoic acid (Atr), 14-aminotetradecanoic acid (Ata), 15-aminopentadecanoic acid (Apn), 16-aminohexadecanoic acid (Ahd), N-(3-aminopropyl)glycine (Apg), (S)-indoline-2-carboxylic acid (Ica), L-α-methyl-leucine (Leu(Me)), and L-2-indanylglycine (Igl), 5-amino-3-oxapentanoic acid (Aea), N-(2-aminoethyl)glycine (Aeg or Aeg2), isonipecotic acid (Inp), 2-cyclohexylglycine, N-butylglycine (ButylGly), N-(4-piperidinyl)glycine (PipGly), 2-amino-3-guanidino-propionic acid (Agp), (4′-pyridyl)alanine (4-PyrAla), (S)—N-(1-phenylethyl)glycine (Feg), N-benzylglycine (Bng), 1,2,3,4-tetrahydroisoquinoline-3-carboxylic acid (Tic), 1,2,3,4-tetrahydroisoquinoline-1-carboxylic acid (Tiq), and 4-guanidino-phenylalanine (Phe(4-Ngu));
with the provisos that when m is 1 and n is 0 or when m is 0 and n is 1, x is absent; when n is 1 and o is 0 or n is 0 and o is 1, y is absent; and when o is 1 and p is 0 or o is 0 and p is 1, z is absent;
optionally, the peptide has a modified N-terminal amino acid in which the N-terminal —NH₂ is replaced by —N(X₁)(X₂), wherein (X₁) and (X₂) are independently selected from H, R₁, R₂C(O), R₃SO₂, and R₄R₅NC(O), wherein R₁, R₂, and R₃ are independently an alkyl group or an alkaryl group, and R₄ and R₅ are independently H, an alkyl group, or an alkaryl group, and wherein the alkyl group and the alkaryl group, independently, are further optionally substituted with halogen, alkyl, amino, and/or oxygen moieties; and
optionally, the peptide has a modified C-terminal amino acid in which the C-terminal —COOH is replaced by —CONH₂ (carboxamide).

In some embodiments, the peptide of Formula I is selected from the peptides in Table 1 (SEQ ID NOs: 2-98).

TABLE 1
Peptides of Formula I

| Seq ID | |
|---|---|
| NO. | Structure* |
| 2 | rRr-G-rRr-G-rRrR-G-rRrR-Cys |
| 3 | rRr-G-rRr-G-rRrR-G-rRrR |
| 4 | rRr-G-rRr-G-rRrR-G-rRrR-NH₂ |
| 5 | rRr-(Ahp)-rRr-(Ava)-rRrR-(Ava)-rRrR-Cys |
| 6 | Cys-rRr-(Ahp)-rRr-(Ava)-rRrR-(Ava)-rRrR |
| 7 | rRr-(Apr)-rRr-(Apr)-rRrR-(Apr)-rRrR-NH₂ |
| 8 | rRr-(Apr)-rRr-(Apr)-rRrR-(Apr)-rRrR |
| 9 | RRR-(Apr)-rRr-(Apr)-rRrR-(Apr)-rRrR-NH₂ |
| 10 | rRr-(Apr)-RRR-(Apr)-rRrR-(Apr)-rRrR-NH₂ |
| 11 | rRr-(Apr)-rRr-(Apr)-RRRR-(Apr)-rRrR-NH₂ |
| 12 | rRr-(Apr)-rRr-(Apr)-rRrR-(Apr)-RRRR-NH₂ |
| 13 | RRR-(Apr)-RRR-(Apr)-RRRR-(Apr)-RRRR-NH₂ |
| 14 | rrr-(Apr)-rrr-(Apr)-rrrr-(Apr)-rrrr-NH₂ |
| 15 | RrR-(Apr)-RrR-(Apr)-RrRr-(Apr)-RrRr-NH₂ |
| 16 | rRr-(Apr)-rRr-(Apr)-rRrR-(Apr)-rRrR-(Aoa) |
| 17 | rRr-(Apr)-rRr-(Apr)-rRrR-(Apr) |
| 18 | rRr-(Ava)-rRr-(Ava)-rRrR-(Ava)-rRrR-NH₂ |
| 19 | rRr-(Ava)-rRr-(Ava)-rRrR-(Ava)-rRrR |
| 20 | rRr-(Ava)-rRr-(Ava)-rRrR-(Ava)-rRrR-Glu |
| 21 | rRrR-(Ava)-rRrR-(Ava)-rRr-(Ava)-rRr-NH₂ |
| 22 | rRr-(Ava)-rRr-(Ava)-rRr-(Ava)-rRr-NH₂ |
| 23 | rR-(Ava)-rRr-(Ava)-rRr-(Ava)-rR-NH₂ |
| 24 | rRr-(Ava)-rR-(Ava)-rRr-(Ava)-rR-NH₂ |
| 25 | rR-(Ava)-rRr-(Ava)-rR-(Ava)-rRr-NH₂ |
| 26 | rR-(Ava)-rR-(Ava)-rRr-(Ava)-rRr-NH₂ |
| 27 | rRr-(Ava)-rRr-(Ava)-rR-(Ava)-rR-NH₂ |
| 28 | rRr-(Ava)-rR-(Ava)-rR-(Ava)-rRr-NH₂ |
| 29 | rRr-(Aoa)-rRr-(Aoa)-rRrR-(Aoa)-rRrR-NH₂ |
| 30 | rRr-(Aoa)-rRr-(Aoa)-rRrR-(Aoa)-rRrR |
| 31 | CH₃CONH-rRr-(Aoa)-rRr-(Aoa)-rRrR-(Aoa)-rRrR-NH₂ |
| 32 | rRr-(Ada)-rRr-(Aoa)-rRrR-(Aoa)-rRrR |
| 33 | rRr-(Ada)-rRr-(Aoa)-rRrR-(Aoa)-rRrR-NH₂ |
| 34 | rRr-(Ada)-rRr-(Aoa)-rRrR-(Aoa)-rRrR-Aoa |
| 35 | rRr-(ButylGly)-rRr-(ButylGly)-rRrR-(ButylGly)-rRrR-N_{H2} |
| 36 | rR-(ButylGly)-rR-(ButylGly)-rRr-(ButylGly)-rRr-NH₂ |
| 37 | rRr-Leu(Me)-rRr-Leu(Me)-rRrR-Leu(Me)-rRrR-NH₂ |
| 38 | rRr-(Apg)-rRr-(Apg)-rRrR-(Apg)-rRrR-NH₂ |
| 39 | rR-(Apg)-rR-(Apg)-rRr-(Apg)-rRr-NH₂ |
| 40 | rRr-(Agp)-rRr-(Agp)-rRrR-(Agp)-rRrR-NH₂ |
| 41 | rRr-(Aeg)-rRr-(Ava)-rRrR-(Ava)-rRrR-NH₂ |
| 42 | rRr-(Ava)-rRr-(Aeg)-rRrR-(Ava)-rRrR-NH₂ |
| 43 | rRr-(Ava)-rRr-(Ava)-rRrR-(Aeg)-rRrR-NH₂ |
| 44 | rRr-(Aeg)-rRr-(Aeg)-rRrR-(Ava)-rRrR-NH₂ |
| 45 | rRr-(Ava)-rRr-(Aeg)-rRrR-(Aeg)-rRrR-NH₂ |
| 46 | rRr-(Aeg)-rRr-(Aeg)-rRrR-(Aeg)-rRrR-NH₂ |
| 47 | rRr-(Aea)-rRr-(Ava)-rRrR-(Ava)-rRrR-NH₂ |
| 48 | rRr-(Ava)-rRr-(Aea)-rRrR-(Ava)-rRrR-NH₂ |
| 49 | rRr-(Ava)-rRr-(Ava)-rRrR-(Aea)-rRrR-NH₂ |
| 50 | rRr-(Aea)-rRr-(Aea)-rRrR-(Ava)-rRrR-NH₂ |
| 51 | rRr-(Ava)-rRr-(Aea)-rRrR-(Aea)-rRrR-NH₂ |
| 52 | rRr-(Aea)-rRr-(Aea)-rRrR-(Aea)-rRrR-NH₂ |
| 53 | rRr-P-rRr-P-rRrR-P-rRrR-NH₂ |
| 54 | rRr-P-rRr-P-rRrR-P-rRrR |
| 55 | CH₃CONH-rRr-P-rRr-P-rRrR-P-rRrR-NH₂ |
| 56 | CH₃SO₂NH-rRr-P-rRr-P-rRrR-P-rRrR-NH₂ |
| 57 | 4-FPhNHC(O)NH-rRr-P-rRr-P-rRrR-P-rRrR-NH₂ |
| 58 | cHexC(O)NH-rRr-P-rRr-P-rRrR-P-rRrR-NH₂ |
| 59 | MorpholineCH₂C(O)NH-rRr-P-rRr-P-rRrR-P-rRrR-NH₂ |
| 60 | rRrR-P-rRrR-P-rRr-P-rRr-NH₂ |
| 61 | rRrRr-P-rRrRr-P-rRrR-NH₂ |
| 62 | rRrRrRr-P-rRrRrRr-NH₂ |
| 63 | RrRr-P-rRr-P-rRrR-P-rRrR-NH₂ |
| 64 | RrRr-P-rRrR-P-rRrR-P-rRrR-NH₂ |
| 65 | RrRr-P-rRr-P-RrR-P-rRr-NH₂ |
| 66 | rRr-P-rRr-P-rRr-P-rRr-NH₂ |
| 67 | rRrR-P-rRrR-P-rRrR-NH₂ |
| 68 | rRr-P-rR-P-rRr-P-rR-NH₂ |
| 69 | rRr-P-rRr-P-rR-P-rR-NH₂ |
| 70 | rRr-P-rR-P-rR-P-rRr-NH₂ |
| 71 | rR-P-rRr-P-rRr-P-rR-NH₂ |
| 72 | rR-P-rRr-P-rR-P-rRr-NH₂ |
| 73 | rR-P-rR-P-rRr-P-rRr-NH₂ |
| 74 | rRr-(Inp)-rRr-(Inp)-rRrR-(Inp)-rRrR-NH₂ |
| 75 | rRr-(Igl)-rRr-(Igl)-rRrR-(Igl)-rRrR-NH₂ |
| 76 | rRr-(Tic)-rRr-(Tic)-rRrR-(Tic)-rRrR-NH₂ |
| 77 | rRr-(4-PyrAla)-rRr-(4-PyrAla)-rRrR-(4-PyrAla)-rRrR-NH₂ |
| 78 | rR-(4-PyrAla)-rR-(4-PyrAla)-rRr-(4-PyrAla)-rRr-NH₂ |
| 79 | rR-(Agp)-rR-(Agp)-rRr-(Agp)-rRr-NH₂ |
| 80 | rRr-(Aeg2)-rRr-(Aeg2)-rRrR-(Aeg2)-rRrR-NH₂ |
| 81 | rRr-(PipGly)-rRr-(PipGly)-rRrR-(PipGly)-rRrR-NH₂ |
| 82 | rRr-(Feg)-rRr-(Feg)-rRrR-(Feg)-rRrR-NH₂ |
| 83 | rRr-(Bng)-rRr-(Bng)-rRrR-(Bng)-rRrR-NH₂ |
| 84 | rR-(Aeg2)-rR-(Aeg2)-rRr-(Aeg2)-rRr-NH₂ |
| 85 | rR-(PipGly)-rR-(PipGly)-rRr-(PipGly)-rRr-NH₂ |
| 86 | rR-(Feg)-rR-(Feg)-rRr-(Feg)-rRr-NH₂ |
| 87 | rR-(Bng)-rR-(Bng)-rRr-(Bng)-rRr-NH₂ |
| 88 | r(Har)r-P-r(Har)r-P-r(Har)r(Har)-P-r(Har)r(Har)-NH₂ |
| 89 | r(Har)r-(Ava)-r(Har)r-(Ava)-r(Har)r(Har)-(Ava)-r(Har)r(Har)-NH₂ |
| 90 | (Har)(Har)(Har)-P-(Har)(Har)(Har)-P-(Har)(Har)(Har)(Har)-P- |
| | (Har)(Har)(Har)(Har)-NH₂ |
| 91 | (Har)(Har)(Har)-(Aoa)-(Har)(Har)(Har)-(Aoa)-(Har)(Har)(Har)(Har)-(Aoa)- |
| | (Har)(Har)r(Har)(Har)-NH₂ |
| 92 | rRr-(Ado)-rRr-(Ado)-rRrR-(Ado)-rRrR-NH₂ |
| 93 | rRr-P-rRr-P-rRrR-P-rRr(hLeu)-NH₂ |
| 94 | (hLeu)Rr-P-rRr-P-rRrR-P-rRrR-NH₂ |
| 95 | (hLeu)Rr-P-rRr-P-rRrR-P-rRr(hLeu)-NH₂ |
| 96 | r(Har)r-(Aoa)-r(Har)r-(Aoa)-r(Har)r(Har)-(Aoa)-r(Har)r(Har)-NH₂ |
| 97 | (Har)R(Har)-(Aoa)-(Har)R(Har)-Aoa-(Har)R(Har)-(Aoa)-(Har)R(Har)R-NH₂ |
| 98 | (Har)R(Har)-P-(Har)R(Har)-P-(Har)R(Har)-P-(Har)R(Har)R-NH₂ |

*Peptides in which the C-terminal -COOH is replaced by a carboxamide are indicated with -NH₂. Peptides in which the N-terminal -NH₂ group having one of the H atoms replaced with another group, the -NH is indicated along with that group e.g., CH₃CONH or CH₃SO₂NH.
4-FPhNHC(O) = 4-fluorophenylaminocarbonyl
cHexC(O) = cyclohexylcarbonyl
MorpholineCH₂C(O) = 2-morpholinoacetyl

| | Chemical | |
|---|---|---|
| Symbol | Name | Structure |
| 4-F—PhNHC(O) | 4-Fluoro- phenylamino- carbonyl | 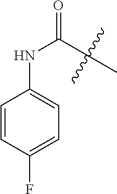 [SMILES: *C(=O)Nc1ccc(F)cc1] |
| cHexNHC(O) | Cyclo- hexylamino- carbonyl | 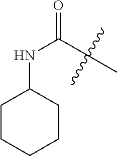 [SMILES: *C(=O)NC1CCCCC1] |
| MorpholineCH₂C(O) | 2-Morpho- linoacetyl | 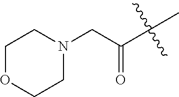 [SMILES: *C(=O)CN1CCOCC1] |
| CH₃SO₂ | Methane- sulfonyl | 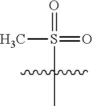 [SMILES: *S(C)(=O)=O] |
| CH₃CO | Acetyl | 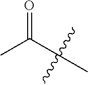 [SMILES: *C(C)=O] |

In another aspect, the present invention provides a peptide-conjugate comprising a peptide of Formula I or Table 1 and a group linked to the C-terminus or N-terminus or the peptide, the group being selected from a polyethylene glycol (PEG) group, a glycosyl group, a lipid group, a cholesterol or sterol group, a peptide or protein group, and an oligonucleotide group.

In another aspect, the present invention provides pharmaceutical compositions comprising a peptide of Formula I or Table 1 or a peptide-conjugate comprising a peptide of Formula I or Table 1 and one or more pharmaceutically acceptable carriers, binders, diluents, and/or excipients.

In another aspect, the present invention provides a method of treating a microbial infection in a subject in need thereof comprising administering to the subject a pharmaceutical composition containing a peptide of Formula I or Table 1 or a peptide-conjugate including a peptide of Formula I or Table 1.

In some embodiments, the microbial infection is a fungal infection. In some embodiments, the infection is with a fungus selected from Absidia spp., Acremonium spp., Actinomadura spp., Apophysomyces spp., Arthrographis spp., Aspergillus spp., Basidiobolus spp., Beauveria spp., Blastomyces spp., Blastoschizomyces spp., Candida spp., Chrysosporium spp., Cladophialophora spp., Coccidioides spp., Conidiobolus spp., Cryptococcus spp., Cunninghamella spp., Emmonsia spp., Epidermophyton spp., Exophiala spp., Fonsecaea spp., Fusarium spp., Geotrichum spp., Graphium spp., Histoplasma spp., Lacazia spp., Leptosphaeria spp., Lomentaspora spp., Malassezia spp., Microsporum spp., Mucor spp., Neotestudina spp., Nocardia spp., Nocardiopsis spp., Paecilomyces spp., Paracoccidiomyces spp., Phialophora spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Sporotrix spp., Syncephalastrum spp., Tinea spp., Trichoderma spp., Trichophyton spp., Trichosporon spp., Ulocladium spp., Ustilago spp., Verticillium spp., and Wangiella spp. In some embodiments, the method further comprises administering another antifungal agent to the subject.

In some embodiments, the microbial infection is a bacterial infection. In some embodiments, the infection is with a gram-positive bacteria, gram-negative bacteria, or mycobacteria. For example, the bacterial can be Enterococcus faecium, Staphylococcus aureus, Escherichia coli, Klebsiella pneumoniae, Pseudomonas aeruginosa, Salmonella senftenberg, Shigella sonnei, or Mycobacterium spp.

The details of one or more embodiments are set forth in the accompanying drawing and the description below. Other features, objects, and advantages of the embodiments will be apparent from the description and drawing, and from the claims. All publications cited herein are hereby incorporated herein by reference.

### BRIEF DESCRIPTION OF THE FIGURES

FIG. 1 is a set of graphs showing time-kill kinetics of SEQ ID NO: 7 and SEQ ID NO: 8 in Candida albicans ATCC 90028 (A) and Cryptococcus neoformans ATCC MYA-4564 (B). CFU is colony forming unit and MIC is minimum inhibitory concentration.

FIG. 2 is a graph showing the mean plasma concentrations following single intravenous (IV) and intraperitoneal (IP) doses of SEQ ID NO: 7.

### DETAILED DESCRIPTION OF THE INVENTION

### Abbreviations and Definitions

The term “amino acid” as used herein is understood to mean an organic compound containing both a basic amino group and an acidic carboxyl group. Included within this term are traditional alpha amino acids (e.g., L-amino acids), isomers of alpha amino acids (e.g. D-amino acids) and known amino acids. Alpha amino acids include, but are not limited to, alanine [Ala (3-letter abbreviation); A (1-letter abbreviation)], arginine (Arg; R), asparagine (Asn; N), aspartic acid (Asp; D), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V); homoarginine (Har), homoleucine (hLeu), S-indoline-2-carboxylic acid (Ica), L-α-methyl-leucine (Leu(Me)), and L-2-indanylglycine (Igl), and L-2-cyclohexylglycine.

The term “amine acid” as used herein include 3-aminopropionic acid (β-alanine, Apr), 4-aminobutyric acid (Aba), 5-aminovaleric acid (Ava), 6-aminohexanoic acid (Ahx), 7-aminoheptanoic acid (Ahp), 8-aminooctanoic acid (Aoa), 9-aminononanoic acid (Ana), 10-aminodecanoic acid (Ada), 11-aminoundecanoic acid (Aun), 12-aminododecanoic acid (Ado), 13-aminotridecanoic acid (Atr), 14-aminotetradecanoic acid (Ata), 15-aminopentadecanoic acid (Apn), 16-aminohexadecanoic acid (Ahd), N-(3-aminopropyl)glycine (Apg), (S)-indoline-2-carboxylic acid (Ica), L-α-methyl-leucine (Leu(Me)), and L-2-indanylglycine (Igl), 5-amino-3-oxapentanoic acid (Aea), N-(2-aminoethyl)glycine (Aeg or Aeg2), isonipecotic acid (Inp), 2-cyclohexylglycine, N-butylglycine (ButylGly), N-(4-piperidinyl)glycine (PipGly), 2-amino-3-guanidino propionic acid (Agp), (4′-pyridyl)alanine (4-PyrAla), (S)—N-(1-phenylethyl)glycine (Feg), N-benzylglycine (Bng), 1,2,3,4-tetrahydroisoquinoline-3-carboxylic acid (Tic), 1,2,3,4-tetrahydroisoquinoine-1-carboxylic acid (Tiq), and 4-guanidino-phenylalanine (Phe(4-Ngu)).

The terms “linker” or “linking” refers to a single amino acid or amine acid as defined above (under Formula 1) between any two arginine and/or homoarginine blocks.

The term “peptide” as used herein means, in general terms, a plurality of amino acid residues, and/or amine acids, joined together by peptide bonds. It is used interchangeably and means the same as polypeptide and protein. The term includes a peptide containing a modified C-terminus or N-terminus.

The term “arginine-containing peptide” (ACP) refers to a peptide comprising 6 to 30 amino acid residues of predominantly arginine and/or homoarginine in “blocks” and further comprising “linkers” to connect the blocks. The ACP may also be conjugated at the C-terminus or N-terminus to a polyethylene glycol (PEG), a glycosyl group, a lipid group, a cholesterol or sterol group, a peptide or protein group, and/or an oligonucleotide group. In general, the ACPs of the present invention are considered to be linear peptides.

The ACPs of the present invention are useful, inter alia, as an antimicrobial peptide, for example, against bacteria, fungi, yeast, parasites, protozoa and viruses. The term “antimicrobial peptide” can be used herein to define any peptide that has microbicidal and/or microbistatic activity and encompasses, non-exclusively, any peptide described as having anti-bacterial, anti-fungal, anti-mycotic, anti-parasitic, anti-protozoal, antiviral, anti-infectious, anti-infective and/or germicidal, algicidal, amoebicidal, microbicidal, bactericidal, fungicidal, parasiticidal, and protozoacidal properties.

The term “mycosis” refers to an infectious disease caused by pathogenic fungus in humans and animals. Mycoses are common and a variety of environmental and physiological conditions can contribute to the development of fungal diseases.

The term “candidiasis” refers to a fungal infection caused by a yeast (a type of fungus) of the family Candida. Some species of Candida can cause infection in people; the most common is Candida albicans. Candida normally lives on the skin and inside the body, in places such as the mouth, throat, gut, vagina, and nails without causing any problems. However, it is an opportunistic pathogen and can cause infection if it overgrows or if it invades the bloodstream or certain internal organs such as the brain, lungs, kidney or heart.

The term “minimum inhibitory concentration” (MIC) refers to the lowest concentration of a therapeutic agent which prevents visible growth of microorganisms, especially fungi or bacteria in the case of ACPs of Formula I.

The term “treating” or “treatment” refers to administration of an effective amount of a therapeutic agent to a subject in need thereof with the purpose of curing, alleviating, relieving, remedying, ameliorating, or preventing the disease, the symptoms of it, or the predisposition towards it. Such a subject can be identified by a health care professional based on results from any suitable diagnostic method.

The terms “administer,” “administering”, “administration,” and the like, as used herein, refer to the methods that may be used to enable delivery of agents or compositions to the desired site of biological action.

The term “subject” refers to an animal, preferably a mammal, and most preferably a human, who is the object of treatment, prevention, observation or experiment. Exemplary mammals include mice, rats, rodents, hamsters, gerbils, rabbits, guinea pigs, dogs, cats, sheep, goats, pigs, cows, horses, giraffes, platypuses, primates, such as monkeys, chimpanzees, apes, and humans. In addition, the subject can be a bird including chickens and turkeys.

The phrase “pharmaceutically acceptable” is employed herein to refer to those agents, materials, compositions, and/or dosage forms which are, within the scope of sound medical judgment, suitable for use in contact with the tissues of human beings or, as the case may be, an animal without excessive toxicity, irritation, allergic response. or other problem or complication, commensurate with a reasonable benefit/risk ratio.

The term “alkyl” refers to an alkane group missing one hydrogen. The general formula for the attached acyclic alkyl group is CₙH₂ₙ₊₁. Alkyl groups include methyl, ethyl, propyl, butyl, pentyl, hexyl, heptyl, octyl, nonyl, decyl, undecyl, dodecyl, tridecyl, tetradecyl, pentadecyl, and hexadecyl.

The term “alkaryl” refers to an alkyl group that terminates in an aryl or heteroaryl group, optionally substituted, where optionally substituted includes substituted with halogen, alkyl, amino, and/or oxygen moieties.

### Peptides of the Invention

In one aspect, the present invention provides for compounds having the structure of Formula I:

S1-[block-1]ₘ-x-[block-2]ₙ-y-[block-3]ₒ-z-[block-4]ₚ-S2 Formula I
SEQ ID NO: 1
or a pharmaceutically acceptable salt thereof, wherein
m, n, o and p independently are 0 or 1, with 0 representing absent and 1 representing present, wherein at least two of m, n, o and p are 1;
block-1, block-2, block-3, and block-4 independently comprise 2 to 7 (i.e., 1, 2, 3, 4, 5, 6, or 7) amino acids each independently selected from an L-arginine (R), D-arginine (r) and homoarginine (Har);
S1 and S2 are each independently an amino acid or amine acid other than an R, r or Har, and are independently present or absent;
x, y, and z are each a linker, and each linker is, independently, present or absent and comprised of a single amino acid or amine acid selected from:
proline (P), glycine (G), 3-aminopropionic acid (β-alanine, Apr), 4-aminobutyric acid (Aba), 5-aminovaleric acid (Ava), 6-aminohexanoic acid (Ahx), 7-aminoheptanoic acid (Ahp), 8-aminooctanoic acid (Aoa), 9-aminononanoic acid (Ana), 10-aminodecanoic acid (Ada), 11-aminoundecanoic acid (Aun), 12-aminododecanoic acid (Ado), 13-aminotridecanoic acid (Atr), 14-aminotetradecanoic acid (Ata), 15-aminopentadecanoic acid (Apn), 16-aminohexadecanoic acid (Ahd), N-(3-aminopropyl)glycine (Apg), (S)-indoline-2-carboxylic acid (Ica), L-α-methyl-leucine (Leu(Me)), and L-2-indanylglycine (Igl), 5-amino-3-oxapentanoic acid (Aea), N-(2-aminoethyl)glycine (Aeg or Aeg2), isonipecotic acid (Inp), 2-cyclohexylglycine, N-butylglycine (ButylGly), N-(4-piperidinyl)glycine (PipGly), 2-amino-3-guanidino-propionic acid (Agp), (4′-pyridyl)alanine (4-PyrAla), (S)—N-(1-phenylethyl)glycine (Feg), N-benzylglycine (Bng), 1,2,3,4-tetrahydroisoquinoline-3-carboxylic acid (Tic), 1,2,3,4-tetrahydroisoquinoline-1-carboxylic acid (Tiq), and 4-guanidino-phenylalanine (Phe(4-Ngu));
with the provisos that when m is 1 and n is 0 or when m is 0 and n is 1, x is absent; when n is 1 and o is 0 or n is 0 and o is 1, y is absent; and when o is 1 and p is 0 or o is 0 and p is 1, z is absent;
optionally, the peptide has a modified N-terminal amino acid in which the N-terminal —NH₂ is replaced by —N(X₁)(X₂), wherein (X₁) and (X₂) are independently selected from H, R₁, R₂C(O), R₃SO₂, and R₄R₅NC(O), wherein R₁, R₂, and R₃ are independently an alkyl group or an alkaryl group, and R₄ and R₅ are independently H, an alkyl group, or an alkaryl group, and wherein the alkyl group and the alkaryl group, independently, are further optionally substituted with halogen, alkyl, amino, and/or oxygen moieties; and
optionally, the peptide has a modified C-terminal amino acid in which the C-terminal —COOH is replaced by —CONH₂ (carboxamide).

In some embodiments, the peptides disclosed herein may be provided as a pharmaceutically acceptable salt thereof. The term “pharmaceutically acceptable salt” refers to a salt of a compound that does not cause significant irritation or toxicity to an organism to which it is administered and does not abrogate the biological activity and properties of the peptide. In some embodiments, the salt is an acid addition to the peptide. Pharmaceutical salts can be obtained by reacting a peptide with mineral or organic acids such as hydrochloric acid, hydrobromic acid, acetic acid, methane sulfonic acid, phosphoric acid, mesylate, oxalic acid and the like.

Table 2 shows the chemical structure of representative amine acid linkers of the present invention.

TABLE 2
Chemical Structures of Representative Amine Acid Linkers

| Symbol | Linker Name | Structure |
|---|---|---|
| Apr | 3-aminopropionoic acid (β-Alanine) | 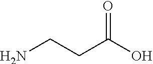 |
| Aba | 4-aminobutyric acid | 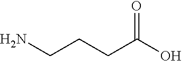 |
| Ava | 5-aminovaleric acid | 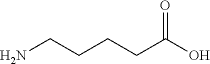 |
| Ahx | 6-aminohexanoic acid | 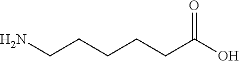 |
| Ahp | 7-aminoheptanoic acid | 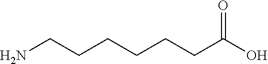 |
| Aoa | 8-aminooctanoic acid | 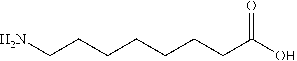 [SMILES: NCCCCCCCC(=O)O] |
| Ana | 9-aminononanoic acid | |
| Ada | 10-aminodecanoic acid | 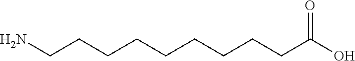 [SMILES: NCCCCCCCCCC(=O)O] |
| Aun | 11-aminoundecanoic acid | 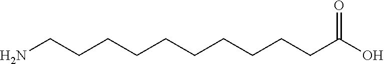 [SMILES: NCCCCCCCCCCC(=O)O] |
| Ado | 12-aminododecanoic acid | 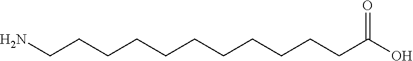 [SMILES: NCCCCCCCCCCCC(=O)O] |
| Atr | 13-aminotridecanoic acid | 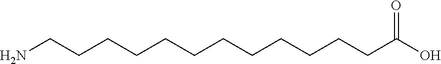 [SMILES: NCCCCCCCCCCCCC(=O)O] |
| Ata | 14-aminotetradecanoic acid | 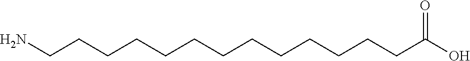 [SMILES: NCCCCCCCCCCCCCC(=O)O] |
| Apn | 15-aminopentadecanoic acid | 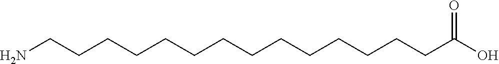 [SMILES: NCCCCCCCCCCCCCCC(=O)O] |
| Ahd | 16-aminohexadecanoic acid | 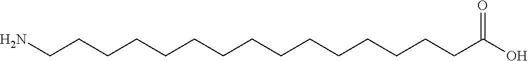 [SMILES: NCCCCCCCCCCCCCCCC(=O)O] |
| Apg | N-(-aminopropyl) | |
| | glycine | |
| Aea | 5-amino-3- | |
| | oxapentanoic acid | |
| Aeg | N-(2-amino- ethyl)glycine | 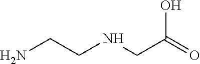 |
| | | Couple at terminal amine |
| Aeg2 | N-(2- aminoethyl)glycine | 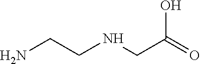 |
| | | Couple at 3-position |
| Ica | L-Indoline-2- carboxylic acid | 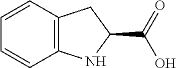 [SMILES: O=C(O)[C@@H]1Cc2ccccc2N1] |
| Igl | L-Indanylglycine | 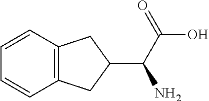 [SMILES: N[C@H](C(=O)O)C1Cc2ccccc2C1] |
| Inp | Isonipecotic acid | 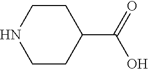 |
| Tic | 1,2,3,4- tetrahydroisoquinoline- 3-carboxylic acid | 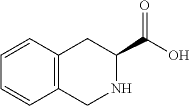 [SMILES: O=C(O)[C@@H]1Cc2ccccc2CN1] |
| ButylGly | N-Butylglycine | 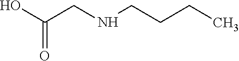 |
| PipGly | N-(4-piperidinyl) glycine | 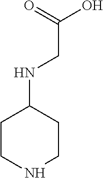 [SMILES: O=C(O)CNC1CCNCC1] |
| | | Couple at 3-position |
| Agp | 2-amino-3-guanidino propionic acid | 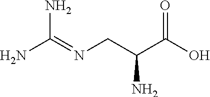 |
| 4-PyrAla | (4′-pyridyl)alanine | 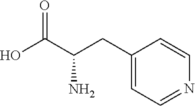 [SMILES: N[C@@H](Cc1ccncc1)C(=O)O] |
| Feg | (S)-N-(1- phenylethyl)glycine | 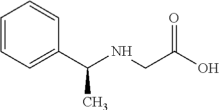 |
| Bng | N-benzylglycine | 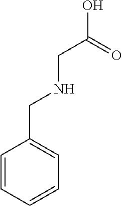 [SMILES: O=C(O)CNCc1ccccc1] |
| Tiq | 1,2,3,4- tetrahydroisoquinoline- 1-carboxylic acid | 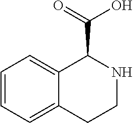 [SMILES: O=C(O)[C@H]1NCCc2ccccc21] |
| Phe(4- Ngu) | 4-guanidino- phenylalanine | 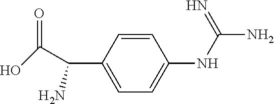 [SMILES: N=C(N)Nc1ccc([C@H](N)C(=O)O)cc1] |
| Leu(Me) | L-α-methyl-leucine | 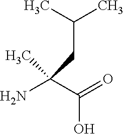 |

In another aspect, the peptide of Formula I is selected from the ACPs in Table 1.

### Biology

In the preferred aspect of the invention the microbial infection may be a fungal infection. The fungal infection may be an infection by Candida spp., (e.g. Candida albicans, Candida glabrata, Candida tropicalis, Candida parapsilosis, Candida krusei, Candida auris, Candida dubliniensis, Candida lusitaniae, Candida guilliermondii), Cryptococcus neoformans, Cryptococcus gatti, Fusarium spp., Scedosporium spp., including Lomentospora prolificans, Coccidioides spp., Trichophyton spp., Microsporum spp., Epidermophyton spp., Aspergillus spp., mucoromycetes, including Rhizopus arrhizus, and/or another fungal species. However, the ACPs may provide treatment against other fungi, such as Exophiala spp., Tinea spp., Blastomyces spp., Blastoschizomyces spp., Cryptococcus spp., Histoplasma spp., Paracoccidiomyces spp., Sporotrix spp., Absidia spp., Cladophialophora spp., Fonsecaea spp., Phialophora spp., Lacazia spp., Arthrographis spp., Acremonium spp., Actinomadura spp., Apophysomyces spp., Emmonsia spp., Basidiobolus spp., Beauveria spp., Chrysosporium spp., Conidiobolus spp., Cunninghamella spp., Geotrichum spp., Graphium spp., Leptosphaeria spp., Malassezia spp. (e.g Malassezia furfur), Mucor spp., Neotestudina spp., Nocardia spp., Nocardiopsis spp., Paecilomyces spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scopulariopsis spp., Sporobolomyces spp., Syncephalastrum spp., Trichoderma spp., Trichosporon spp., Ulocladium spp., Ustilago spp., Verticillium spp., or Wangiella spp.

Invasive candidiasis is an infection caused by a yeast (a type of fungus) called Candida. Unlike Candida infections in the mouth and throat (oropharyngeal candidiasis also called “thrush”) or vaginal (vulvovaginal candidiasis or “yeast infections”), invasive candidiasis is a serious infection that can affect the blood, heart, brain, kidneys, eyes, bones, and other parts of the body. Candidemia, a bloodstream infection with Candida, is a common infection in hospitalized patients.

Cryptococcus is an invasive fungus that causes cryptococcosis an infection commonly associated with immunosuppressed individuals while being rare in healthy individuals. The two species of Cryptococcus that are commonly associated with infections in humans are Cryptococcus neoformans and Cryptococcus gatti. Cryptococcus can infect the meninges to produce cryptococcal meningitis.

Without being bound by any theory, it is hypothesized that the net negative charge of the cell wall and cell membranes of microorganisms may facilitate interaction with the net positive charge of the ACPs, causing cell wall and/or membrane lysis and death, akin to the actions of antimicrobial peptides.

The ACPs of the present invention are positively charged cationic peptides useful for treating infections or diseases caused by a wide variety of pathogenic yeast, molds, bacteria and other microbes. The peptides of the invention may also be useful in the treatment of other conditions including, but not limited to, conditions associated with mucosal infections, for example, cystic fibrosis, gastrointestinal, urogenital, urinary (e.g. kidney infection or cystitis), vaginal or respiratory infections.

### Antifungal Activity

Table 3 shows minimum inhibitory concentrations (MIC) for selected peptides listed in Table 1 tested against various Candida and Cryptococcus species (see Example 2). The peptides were found to possess potent antifungal activity in Candida and Cryptococcus species including against strains resistant to current antifungal therapies as compared to positive reference compounds fluconazole, caspofungin and amphotericin B.

TABLE 3
Peptides from Table 1 and Their Antifungal Activities against Candida and Cryptococcus Species

| | MIC (μg/mL) | | | | | | | | |
|---|---|---|---|---|---|---|---|---|---|
| SEQ | | | | | | | | C. | |
| ID | C. | C. | C. | C. | C. | C. | C. | neo- | C. |
| NO. | albicans | glabrata | parapsilosis | krusei | auris | tropicalis | dubliniensis | formans | gattii |
| 2 | 0.5 | 0.5 | 0.25 | 0.5 | 2 | 0.25 | 0.5 | 0.5 | 0.5 |
| 3 | 0.5 | 0.25 | 0.25 | 1 | 4 | 0.25 | 0.25 | 0.5 | 0.5 |
| 4 | 0.5 | 0.25 | 0.25 | 0.5 | 8 | 0.25 | 0.25 | 0.25 | 0.5 |
| 5 | 1 | 0.5 | 0.25 | 1 | 0.25 | 0.25 | | 1 | 0.5 |
| 6 | 8 | 0.5 | 2 | 8 | 4 | 1 | | | 1 |
| 7 | 0.5 | 0.5 | 0.12 | 0.5 | 1 | 0.25 | 0.25 | 0.25 | 0.25 |
| 8 | 0.5 | 0.5 | 0.25 | 0.5 | 4 | 0.25 | 0.25 | 0.5 | 0.25 |
| 9 | 0.5 | 2 | 0.5 | | | 0.5 | 0.5 | | |
| 10 | 0.5 | 2 | 0.5 | | | 0.5 | 0.5 | | |
| 11 | 1 | >32 | 0.5 | | | 2 | 0.5 | | |
| 12 | 0.5 | >32 | 0.5 | | | 1 | 0.5 | | |
| 13 | 8 | 32 | 0.25 | 2 | 4 | 0.5 | 2 | 1 | 0.5 |
| 14 | 1 | 0.5 | 0.12 | 0.5 | 0.5 | 0.25 | 0.5 | 0.5 | 0.25 |
| 15 | 1 | 0.5 | 0.25 | 0.5 | 0.5 | 0.25 | 0.5 | 0.5 | 0.25 |
| 16 | 1 | 0.5 | 0.5 | 0.5 | 0.5 | 0.5 | 0.5 | 0.5 | 0.25 |
| 17 | 4 | >64 | 2 | 1 | >64 | 4 | 8 | 64 | 8 |
| 18 | 1 | 0.5 | 0.25 | 0.5 | 0.25 | 0.5 | 0.5 | 0.25 | 0.25 |
| 19 | 0.5 | 0.5 | 0.25 | 1 | 2 | 0.5 | 0.5 | | |
| 20 | 0.5 | 1 | 0.25 | 2 | 16 | 0.5 | 0.5 | | |
| 21 | 0.5 | 0.5 | 0.25 | 0.5 | 0.5 | 0.5 | 0.5 | | |
| 22 | 0.5 | 0.5 | 0.25 | 1 | 2 | 0.5 | 0.25 | 1 | 0.5 |
| 23 | 2 | 4 | 0.25 | 4 | >32 | 1 | 1 | 8 | 2 |
| 24 | 2 | 8 | 0.25 | 8 | >32 | 1 | 1 | 16 | 4 |
| 25 | 4 | 8 | 0.5 | 16 | >32 | 2 | 2 | 32 | 4 |
| 26 | 8 | 8 | 0.5 | 8 | >32 | 2 | 4 | 16 | 4 |
| 27 | 2 | 8 | 0.25 | 8 | >32 | 1 | 1 | 8 | 2 |
| 28 | 2 | 4 | 0.5 | 8 | >32 | 1 | 1 | 8 | 4 |
| 29 | 1 | 0.5 | 0.5 | 0.5 | 0.5 | 0.5 | 0.5 | 0.5 | 0.5 |
| 30 | 1 | 1 | 0.5 | | | 1 | 0.5 | | |
| 31 | 1 | 1 | 0.25 | 0.5 | 0.5 | 0.5 | 1 | 0.5 | 0.5 |
| 32 | 1 | 0.5 | 0.25 | 0.5 | 4 | 0.5 | 1 | 0.25 | 0.25 |
| 33 | 1 | 0.5 | 0.5 | 0.5 | 0.5 | 0.5 | 0.25 | 0.25 | 0.5 |
| 34 | 2 | 8 | 0.5 | 1 | 0.5 | 0.5 | 0.5 | 0.5 | 0.5 |
| 35 | 0.5 | 0.5 | 0.25 | 0.5 | 0.5 | 0.5 | 0.5 | | |
| 36 | 0.5 | 4 | 16 | | | 2 | 2 | | |
| 37 | 0.5 | 0.5 | 0.5 | 1 | 1 | 0.5 | 0.5 | | |
| 38 | 0.5 | 1 | 0.25 | 0.25 | 2 | 0.5 | 0.5 | | |
| 39 | 0.25 | 8 | 0.12 | | | 0.25 | 0.12 | | |
| 41 | 1 | 2 | 1 | | | 1 | 0.5 | | |
| 42 | 1 | 1 | 0.5 | | | 0.5 | 0.5 | | |
| 43 | 1 | 1 | 0.5 | | | 0.5 | 0.5 | | |
| 46 | 0.5 | 1 | 0.25 | 0.5 | 8 | 0.5 | 0.5 | | |
| 47 | 1 | 1 | 0.5 | | | 0.5 | 0.5 | | |
| 48 | 0.5 | 1 | 0.5 | | | 0.5 | 0.5 | | |
| 49 | 1 | 1 | 0.5 | | | 0.5 | 0.5 | | |
| 51 | 0.5 | 1 | 0.25 | | | 0.5 | 0.25 | | |
| 52 | 0.5 | 0.5 | 0.25 | 0.5 | 1 | 0.5 | 0.5 | | |
| 53 | 0.5 | 0.5 | 0.25 | 0.5 | 0.25 | 0.5 | 0.25 | 0.5 | 0.25 |
| 54 | 0.25 | 0.5 | 0.25 | 0.5 | 2 | 0.25 | 0.5 | | |
| 55 | 1 | 0.5 | 0.25 | 2 | 1 | 0.5 | 0.5 | 0.5 | 1 |
| 56 | 0.5 | 0.5 | 0.25 | 1 | 0.5 | 0.5 | 0.5 | | |
| 57 | 0.5 | 0.5 | 0.5 | 1 | 0.5 | 0.5 | 0.5 | | |
| 58 | 1 | 1 | 0.5 | 2 | 1 | 1 | 0.5 | | |
| 59 | 0.5 | 0.5 | 0.5 | 2 | 4 | 0.5 | 0.5 | | |
| 60 | 0.25 | 0.25 | 0.25 | 0.5 | 0.5 | 0.25 | 0.25 | | |
| 61 | 0.25 | 0.5 | 0.25 | 0.5 | 1 | 0.25 | 0.25 | | |
| 62 | 0.25 | 0.5 | 0.25 | 0.5 | 2 | 0.25 | 0.25 | | |
| 63 | 0.5 | 0.5 | 0.5 | 0.5 | 0.5 | 0.5 | 0.5 | | |
| 64 | 0.5 | 0.5 | 0.5 | 0.5 | 0.5 | 0.5 | 0.5 | | |
| 65 | 0.25 | 0.5 | 0.25 | 0.5 | 0.5 | 0.5 | 0.5 | | |
| 66 | 0.25 | 0.5 | 0.25 | 0.5 | 8 | 0.25 | 0.25 | 0.5 | 1 |
| 67 | 0.25 | 0.5 | 0.25 | 0.5 | 4 | 0.5 | 0.25 | | |
| 68 | 0.25 | 2 | 0.5 | 2 | >32 | 1 | 0.5 | 8 | 2 |
| 69 | 0.25 | 2 | 0.5 | 1 | >32 | 0.5 | 0.25 | 8 | 2 |
| 70 | 0.5 | 4 | 2 | 2 | >32 | 1 | 1 | 8 | 4 |
| 71 | 0.5 | 1 | 0.25 | 2 | >32 | 0.5 | 1 | 8 | 4 |
| 72 | 2 | 1 | 0.25 | 2 | >32 | 0.5 | 2 | 16 | 4 |
| 73 | 1 | 1 | 0.5 | 2 | >32 | 1 | 2 | 8 | 2 |
| 74 | 0.5 | 0.5 | 0.25 | 1 | 0.5 | 0.5 | 0.5 | | |
| 75 | 0.5 | 0.5 | 0.5 | 0.5 | 0.5 | 0.5 | 0.5 | | |
| 76 | 0.5 | 0.5 | 0.5 | 1 | 0.5 | 0.5 | 0.5 | | |
| 77 | 0.5 | 0.5 | 0.25 | 0.5 | 0.5 | 0.5 | 0.5 | | |
| 78 | 0.5 | 0.5 | 0.25 | 1 | 16 | 0.5 | 0.25 | | |
| 88 | 0.5 | 0.5 | 0.25 | 0.5 | 0.5 | 0.5 | 0.5 | 0.5 | 0.5 |
| 89 | 1 | 1 | 0.5 | 1 | 0.5 | 1 | 1 | 0.25 | 0.25 |
| 93 | 0.5 | 1 | 1 | | | 0.5 | 0.5 | | |
| 94 | 0.5 | 0.5 | 0.25 | 1 | 1 | 0.5 | 0.5 | | |
| 95 | 1 | 0.5 | 1 | | | 1 | 0.5 | | |
| FLC | 0.25 | 4 | 2 | 8 | >32 | 0.25 | 0.5 | 4 | 8 |
| CAS | 0.03 | 0.06 | 0.25 | 0.25 | 0.06 | 0.03 | | 16 | 16 |
| AMB | 0.5 | 0.5 | 0.25 | 0.5 | 1 | 0.5 | 0.25 | 0.25 | 0.5 |

Activity in a typical strain of each Candida or Cryptococcus species (ATCC 90028 or 90029 for C. albicans, ATCC 90030 for C. glabrata, ATCC 22019 for C. parapsilosis, MMX 7135 for C. krusei, CDC 386 for C. auris, ATCC 90874 for C. tropicalis, ATCC MYA-578 for C. dubliniensis, ATCC 90112 for C. neoformans, and ATCC MYA-4093 for C. gattii. MIC: minimum inhibitory concentration; FLC: fluconazole; CAS: caspofungin; AMB: amphotericin B.

Table 4 shows MIC values for selected peptides of Table 1 tested against Coccidioides species (see Example 2). The peptides were found to possess potent antifungal activity in Coccidioides as compared to positive reference compound fluconazole.

TABLE 4
Peptides from Table 1 and Their Antifungal Activities against Coccidioides Species
MIC Range (μg/mL)

| N = 3 | | | | | | | N = 10 | | |
|---|---|---|---|---|---|---|---|---|---|
| Seq ID | SEQ ID | SEQ ID | SEQ ID | SEQ ID | SEQ ID | | SEQ ID | SEQ ID | |
| NO: 32 | NO: 3 | NO: 4 | NO: 7 | NO: 8 | NO: 16 | FLC | NO: 29 | NO: 53 | FLC |
| 0.25-0.5 | 0.25-0.5 | 0.25-0.5 | 0.25-0.5 | 0.25-0.5 | 0.5 | 8->64 | 0.06-0.25 | 0.06-0.25 | 4->64 |

Coccidioides species include C. immitis and C. posadasii. MIC: minimum inhibitory concentration; N: Number of strains; FLC: fluconazole

Table 5 shows MIC values for selected peptides of Table 1 tested against filamentous fungi (see Example 2). The peptides were found to possess potent antifungal activity in these species as compared to positive reference compounds fluconazole, voriconazole, posaconazole, caspofungin, and amphotericin B.

TABLE 5
Peptides from Table 1 and Their Antifungal
Activities against Filamentous Fungi

| | MIC Range (μg/mL) | | | | |
|---|---|---|---|---|---|
| | Fusarium | Scedosporium | Lomentospora | Rhizopus | Paecilomyces |
| SEQ ID | spp. | spp. | prolificans | arrhizus | variotii |
| NO. | (N = 3) | (N = 2) | (N = 1) | (N = 3) | (N = 1) |
| 5 | 0.5-4 | | | | |
| 6 | 0.25-0.5 | | | | |
| 2 | 0.12-0.25 | | | | |
| 34 | 0.25-2 | | | | |
| 32 | 0.25-1 | ≤0.12 | 0.25 | 0.5-8 | 0.25 |
| 3 | 0.12-0.5 | ≤0.12 | ≤0.12 | 1-8 | 0.25 |
| 4 | 0.06-8 | ≤0.12 | ≤0.12 | 0.5-8 | 0.25 |
| 7 | 0.12-0.5 | ≤0.12 | ≤0.12 | 1-8 | 0.25 |
| 8 | 0.25-0.5 | ≤0.12 | ≤0.12 | 2-32 | 0.25 |
| 16 | 0.25-1 | ≤0.12-0.25 | 0.25 | 2-64 | 0.25 |
| 17 | 16-64 | | | | |
| 13 | 0.5-2 | 0.25 | | | |
| 14 | 0.12-0.25 | 0.25 | | | |
| 15 | 0.12-0.25 | 0.25-0.5 | | | |
| 29 | 0.5 | 0.5 | 0.5 | | |
| 18 | 0.25-0.5 | 0.25-0.5 | 0.5 | | |
| 53 | 0.25 | 0.25 | 0.12 | | |
| 33 | 0.25-0.5 | 0.25 | | | |
| 31 | 0.25-4 | 0.5-2 | 2 | | |
| 55 | 0.25-1 | 0.5-1 | 1 | | |
| 66 | 0.12-8 | 0.5-2 | 2 | | |
| 71 | 2->32 | 4-8 | 8 | | |
| 68 | 2->32 | 8 | >32 | | |
| 72 | 8->32 | 16 | 32 | | |
| 73 | 2->32 | 8-16 | 32 | | |
| 69 | 4->32 | 8-16 | 32 | | |
| 70 | 4->32 | 8 | 32 | | |
| 22 | 0.5-16 | 1-2 | 2 | | |
| 23 | 4->32 | 16-32 | >32 | | |
| 24 | 8->32 | 8-32 | >32 | | |
| 25 | 16->32 | 16-32 | >32 | | |
| 26 | 8->32 | 16-32 | 32 | | |
| 27 | 4->32 | 8-16 | 32 | | |
| 28 | 2->32 | 4 | 32 | | |
| FLC | >32 | 8->32 | | | |
| VOR | 4-16 | 1-2 | 16 | | 0.12 |
| POS | | | | 0.12-0.25 | ≤0.03 |
| CAS | 32 | 32 | | | |
| AMB | 1 | 1-2 | | | |

Scedosporium species include S. boydii and S. apiospermum. MIC: minimum inhibitory concentration; N: number of strains; FLC: fluconazole; VOR: voriconazole; POS: posaconazole; CAS: caspofungin; AMB: amphotericin B

Table 6 shows MIC values for SEQ ID NO: 5, SEQ ID NO: 2, SEQ ID NO: 34, and SEQ ID NO: 32 tested against three dermatophyte species (see Example 2). The peptides were found to possess potent antifungal activity in these species as compared to positive reference compounds fluconazole, caspofungin and amphotericin B.

TABLE 6
Peptides from Table 1 and Their Antifungal Activities against Dermatophytes

| | MIC (μg/mL) | | | | | | |
|---|---|---|---|---|---|---|---|
| | SEQ ID | SEQ ID | SEQ ID | SEQ ID | | | |
| Species & Strain | NO: 5 | NO: 2 | NO: 34 | NO: 32 | FLC | CAS | AMB |
| Trichophyton rubrum | 2 | 2 | 4 | 8 | 0.5 | 2 | ≤0.06 |
| ATCC MYA-4438 | | | | | | | |
| Epidermophyton | 16 | 4 | 8 | 4 | 1 | 0.06 | 0.12 |
| floccosum ATCC 52066 | | | | | | | |
| Microsporum gypseum | 64 | >64 | 32 | >64 | 2 | 0.12 | 0.5 |
| ATCC 14683 | | | | | | | |

MIC: minimum inhibitory concentration;
FLC: fluconazole;
CAS: caspofungin;
AMB: amphotericin B

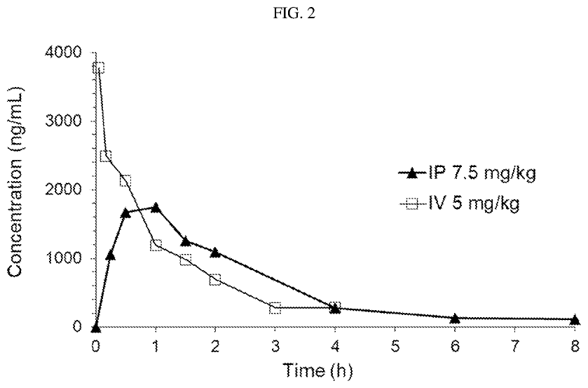

The ACPs exhibit rapid fungicidal activity (defined as a 3-log drop in CFU/mL from time zero in a time-kill kinetics assay) as shown by SEQ ID NO: 7, SEQ ID NO: 8 and SEQ ID NO: 32 in C. albicans (FIG. 2A) and in C. neoformans (FIG. D).

### Antibacterial Activity

Table 7 shows MIC values for selected peptides of Table 1 tested against gram-positive and gram-negative bacterial species as well as mycobacteria species (see Example 4).

TABLE 7
Peptides from Table 1 and Their Antibacterial Activities

| | MIC (μg/mL) | | | | | | | | | |
|---|---|---|---|---|---|---|---|---|---|---|
| | Seq | Seq | Seq | Seq | Seq | Seq | Seq | Seq | Seq | Seq |
| Species & Strain | ID 5 | ID 6 | ID 2 | ID 34 | ID 32 | ID 17 | ID 15 | ID 29 | ID 18 | ID 33 |

Gram-Positive Bacteria

| | | | | | | | | | | |
|---|---|---|---|---|---|---|---|---|---|---|
| Enterococcus faecium ATCC | 16 | 16 | | | | | | | | |
| 700221 | | | | | | | | | | |
| Staphylococcus aureus ATCC | 8 | 8 | 8 | 16 | 8 | >64 | 16 | 16 | 16 | 16 |
| 29213 | | | | | | | | | | |

Gram-Negative Bacteria

| | | | | | | | | | | |
|---|---|---|---|---|---|---|---|---|---|---|
| Escherichia coli ATCC 25922 | 16 | 16 | 8 | 4 | 2 | >64 | 32 | 16 | 32 | 16 |
| Klebsiella pneumoniae ATCC | | 16 | 64 | 8 | 4 | | | | | |
| 13883 | | | | | | | | | | |
| Pseudomonas aeruginosa ATCC | 32 | 16 | 32 | 16 | 16 | >64 | 64 | 32 | 64 | 32 |
| 27853 | | | | | | | | | | |
| Salmonella senftenberg CDC 127 | 16 | 16 | 32 | 8 | 4 | | | | | |
| Shigella sonnei MMX 10772 | | 16 | 16 | 4 | 4 | | | | | |

Mycobacteria

| | | | | | |
|---|---|---|---|---|---|
| Mycobacterium fortuitum ATCC | | | 4 | 2 | 2 |
| 110 | | | | | |
| Mycobacterium smegmatis ATCC | 2 | 1 | 1 | 1 | 0.5 |
| 700084 | | | | | |

MIC: minimum inhibitory concentration

### Structure Activity Relationships

The role of a C-terminal cysteine on antifungal and antibacterial activity in arginine-containing peptides (ACPs) was investigated and found not to be required (Tables 3 and 7-8). SEQ ID NO: 2, which contains a C-terminal cysteine has similar activity against Candida spp. to SEQ ID NO: 3, which has the identical sequence except lacking a C-terminal cysteine. In Table 7, only SEQ ID NO: 5 contains a C-terminal cysteine and its MIC values against bacteria are similar to those ACP structures without a cysteine. The addition of a C-terminal cysteine increases cytotoxicity (Table 9).

TABLE 8
Effect of C-Terminal Cysteine on MICs
Against Candida Spp. (N = 25)

| | MIC₅₀ | MIC₉₀ |
|---|---|---|
| Peptide | (μg/mL) | (μg/mL) |
| SEQ ID NO: 2 | 0.5 | 2 |
| SEQ ID NO: 3 | 0.5 | 4 |

Candida species include: 5 strains each of C. albicans, C. auris, and C. glabrata, 3 strains each of C. krusei, C. parapsilosis, and C. tropicalis, and 1 strain of C. dubliniensis. MIC₅₀: the minimum inhibitory concentration at which 50% of fungal strains are inhibited; MIC₉₀: the minimum inhibitory concentration at which 90% of fungal strains are inhibited.

TABLE 9
Effect of C-Terminal Cysteine on Cytotoxicity in HepG2 Cells

| | | | Cytotoxic Reference |
|---|---|---|---|
| | Peptide | | Compound |
| | SEQ ID NO: 2 | SEQ ID NO: 3 | Melittin |
| EC₅₀ | 39.64 | >300 | 2.014 |
| (μg/mL) | | | |

EC₅₀: Concentration at which 50% cell viability is observed. Lower EC₅₀ indicates greater cytotoxicity.

The role of arginine stereochemistry on antifungal activity in ACPs was investigated. The results showed that when all the arginine amino acids in an ACP have the L stereochemistry, ie., L-arginines, the ACP lacks or has significantly reduced antifungal activity (Table 10). The best antifungal activity is found in ACPs where all the arginines have the D-stereochemistry, ie., D-arginines, or APCs having a mixture of L- and D-arginines, or ACPs possessing homoarginines (Har) (Table 10).

TABLE 10
Antifungal Activity of ACPs that have All L, All D, or a
Mixture of L and D Arginines and/or Homoarginines (Har)

| Peptide | MIC₅₀ | MIC₉₀ |
|---|---|---|
| (D/L Arginine Composition) | (μg/mL) | (μg/mL) |
| SEQ ID NO: 13 | 4 | 16 |
| (All L) | | |
| SEQ ID NO: 14 | 0.5 | 2 |
| (All D) | | |
| SEQ ID NO: 7 | 0.5 | 1 |
| (D and L Mix) | | |
| SEQ ID NO: 15 | 0.5 | 1 |
| (D and L Mix) | | |
| SEQ ID NO: 88 | 0.5 | 1 |
| (D-Arg and Har Mix) | | |

Activity screen in 15 strains of Candida species (4 strains of C. auris, 2 strains each of C. albicans, C. glabrata, C. krusei, C. parapsilosis, and C. tropicalis, and 1 strain of C. dubliniensis). MIC₅₀: the minimum inhibitory concentration at which 50% of fungal strains are inhibited; MIC₉₀: the minimum inhibitory concentration at which 90% of fungal strains are inhibited.

The impact of the total number of arginines in an ACP on antifungal or antibacterial activity was investigated. The results showed that optimal activity is found in ACPs with a total of 10 to 16 arginine amino acids containing a mixture of L- and D-arginines. ACPs containing 14 arginine amino acids displayed the lowest MIC₅₀ or MIC₉₀ values (Table 11). Reduced activity is observed in ACPs with less than 12 arginine amino acids in Candida spp. (Table 11) and in Cryptococcus spp. and filamentous fungi (Table 12). ACPs containing 14 arginines possess antibacterial activity (Table 7), but antibacterial activity is abolished in ACPs containing only 10 arginines (SEQ ID NO: 17, Table 7).

TABLE 11
Impact of Number of Arginine Amino Acids
on MICs against Candida Spp. (N = 15)

| Peptide | Number | | Activity at 24 h | | Activity at 48 h | |
|---|---|---|---|---|---|---|
| SEQ ID | of | Linkers | MIC₅₀ | MIC₉₀ | MIC₅₀ | MIC₉₀ |
| NO. | Arginines | (Number) | (μg/mL) | (μg/mL) | (μg/mL) | (μg/mL) |
| 70 | 10 | Proline (3) | 4 | >32 | 8 | >32 |
| 66 | 12 | Proline (3) | 0.5 | 8 | 0.5 | 16 |
| 65 | 13 | Proline (3) | 0.5 | 2 | 0.5 | 32 |
| 53 | 14 | Proline (3) | 0.5 | 0.5 | 0.5 | 2 |
| 63 | 15 | Proline (3) | 0.5 | 1 | 1 | 2 |
| 64 | 16 | Proline (3) | 0.5 | 1 | 0.5 | 2 |
| 28 | 10 | Ava (3) | 8 | >32 | 8 | >32 |
| 22 | 12 | Ava (3) | 1 | 8 | 2 | 32 |
| 18 | 14 | Ava (3) | 0.5 | 1 | 0.5 | 2 |

Candida species include 4 strains of C. auris, 2 strains each of C. albicans, C. glabrata, C. krusei, C. parapsilosis, and C. tropicalis, and 1 strain of C. dubliniensis. MIC₅₀: the minimum inhibitory concentration at which 50% of fungal strains are inhibited; MIC₉₀: the minimum inhibitory concentration at which 90% of fungal strains are inhibited. Ava: 5-aminovaleric acid

TABLE 12
Impact of Number of Arginine Amino Acids on MICs
Against Cryptococcus Spp. and Filamentous Fungi

| | | | MIC Range (μg/mL) | |
|---|---|---|---|---|
| Peptide | | | Cryptococcus | Filamentous |
| SEQ ID | No. of | Linkers | spp. | Fungi |
| NO. | Arginines | (Number) | (N = 3) | (N = 5) |
| 70 | 10 | Proline (3) | 1-8 | 4->32 |
| 66 | 12 | Proline (3) | 0.5-1 | 0.12-8 |
| 53 | 14 | Proline (3) | 0.25-0.5 | 0.12-0.25 |
| 28 | 10 | Ava (3) | 2-8 | 2->32 |
| 22 | 12 | Ava (3) | 0.5-1 | 0.5-16 |
| 18 | 14 | Ava (3) | 0.25-0.5 | 0.25-0.5 |

MIC: minimum inhibitory concentration;
Cryptococcus species include 2 strains of C. neoformans and 1 strain of C. gattii;
Filamentous fungi include 3 strains of Fusarium spp. and 2 strains of Scedosporium spp. (S. boydii and S. apiospermum).

Enhanced antifungal activity is observed when arginines are organized and arranged in 2 to 4 blocks with a linker placed between each of the two blocks of arginines and/or homoarginines (Table 13). Separation of these arginine- and/or homoarginine-containing blocks by a linker between any two blocks of arginines or homoarginines is essential to enhanced antifungal activity, with ACPs containing 3 linkers and 4 arginine and/or homoarginine blocks exhibiting the best activity, ie, lowest MIC₉₀ value (Table 13).

TABLE 13
Impact of Number of Linkers on MICs
against Candida spp. (N = 15)

| | | Number of | | | | |
|---|---|---|---|---|---|---|
| | Number | Arginines | Activity at 24 h | | Activity at 48 h | |
| | of | (No. of | MIC50 | MIC90 | MIC50 | MIC90 |
| Peptide | Linkers | Blocks) | (μg/mL) | (μg/mL) | (μg/mL) | (μg/mL) |
| 14 | 0 | 14 (1) | 0.5 | 8 | 0.5 | >32 |
| Polyarginine | | | | | | |
| SEQ ID | 1 | 14 (2) | 0.5 | 4 | 0.5 | 32 |
| NO: 62 | | | | | | |
| SEQ ID | 2 | 14 (3) | 0.5 | 1 | 0.5 | 16 |
| NO: 61 | | | | | | |
| SEQ ID | 3 | 14 (4) | 0.5 | 0.5 | 0.5 | 2 |
| NO: 53 | | | | | | |

Candida species include 4 strains of C. auris, 2 strains each of C. albicans, C. glabrata, C. krusei, C. parapsilosis, and C. tropicalis, and 1 strain of C. dubliniensis. MIC₅₀: the minimum inhibitory concentration at which 50% of strains are inhibited; MIC₉₀: the minimum inhibitory concentration at which 90% of strains are inhibited; 14 Polyarginine: rRrrRrrRrRrRrR-NH2

### Pharmaceutical Compositions

The term “pharmaceutical composition” refers to a mixture of a therapeutic agent disclosed herein (ie. ACP) with other chemical components, such as pharmaceutically acceptable diluents, carriers, binders and/or excipients. The pharmaceutical composition facilitates administration of the compound to a subject.

The present disclosure relates to a pharmaceutical composition comprising physiologically acceptable carriers, binders, excipients and/or diluents for therapeutic use that are well known in the pharmaceutical art, and are described, for example, in Remington's Pharmaceutical Sciences, 18th Ed., Mack Publishing Co., Easton, Pa. (1990), which is incorporated herein by reference in its entirety.

The pharmaceutical compositions of the present invention may be manufactured in a manner that allows for various routes of administration. Delivery by parenteral administration, e.g., by bolus injection or continuous infusion, include aqueous solutions of the therapeutic agent or suspensions may contain substances which increase the viscosity of the suspension; optionally, the suspension may also contain suitable stabilizers or agents that increase the solubility of the compounds to allow for the preparation of highly concentrated solutions. The therapeutic arginine- and/or homoarginine-containing peptides may be formulated as is known in the art for direct topical application to a target area. For oral administration, the compounds can be formulated readily by combining the active compounds with pharmaceutically acceptable carriers well known in the art to produce tablets, pills, dragees, capsules, sachets, liquids, gels, syrups, lozenges, slurries, suspensions and the like, for oral ingestion by a patient to be treated. Additionally, the peptides may be formulated for administration to the respiratory tract as a nebulized liquid or dry powder, or as drops, such as eye drops or nose drops, or as oral rinse.

### Methods of Administration

Suitable routes of administration may include, but are not limited to, oral, sublingual, transmucosal, inhalation, transdermal, topical, vaginal or rectal administration; parenteral delivery, including intramuscular, subcutaneous, intravenous, intramedullary, or intrathecal injections, as well as intranasal, or intraocular injections. The compounds can also be administered in sustained or controlled release dosage forms, depot formulation, continuous infusion via a pump or pulsed administration at a predetermined rate.

Pharmaceutical compositions suitable for administration include compositions where the active ingredients are contained in a therapeutically effective amount to prevent, alleviate or ameliorate symptoms of disease or prolong the survival of the subject being treated. The determination of effective dosage levels, that is the dosage levels necessary to achieve the desired treatment outcome, can be accomplished by one skilled in the art using routine pharmacological methods. Administration of the therapeutic agents in accordance with the present invention may be in a single dose, in multiple doses, in a continuous or intermittent manner; both systemic and local administrations are contemplated.

### Methods of Treatment

This disclosure provides a therapeutic method of treating a subject suffering from an infection with a fungus or other microbes by administering a compound of the present invention to the subject. Therapeutic treatment is initiated after diagnosis or the development of symptoms of infection with a fungus or other microbe. One or more peptides listed in Table 1 or a peptide of Formula I, or a combination thereof, of the present invention may be used to treat or prevent fungal or microbial infections. Exemplary fungal infections include, but are not limited to, an infection with a Candida species including, for example, Candida albicans, Candida glabrata, Candida tropicalis, Candida parapsilosis, Candida krusei, Candida auris, Candida dubliniensis, Cryptococcus neoformans, Cryptococcus gatti, Fusarium spp., Scedosporium spp., including Lomentospora prolificans, Coccidioides spp., Trichophyton spp., Microsporum spp., Epidermophyton spp., Aspergillus spp., mucoromycetes, including Rhizopus arrhizus, and/or another fungal species.

Preventive or prophylactic antifungal therapy is regularly practiced during the treatment of patients with cancer and high-risk liver transplant recipients (Rex J H, et al., Healthcare Epidemiology CID 2001:32, pages 1191-1200). The principal fungi of concern in these patients or other transplant or immunocompromised patients are Candida species and various filamentous fungi, especially Aspergillus species. Additionally, healthcare providers sometimes prescribe preventive or prophylactic antifungal therapy to patients at high risk for developing invasive candidiasis such as critically ill patients in intensive care units, organ transplant patients, stem cell or bone marrow transplant patients with low white blood cell counts (neutropenia), and in very low weight infants (less than 2.2 pounds) in nurseries with high rates of invasive candidiasis.

A peptide of the present invention may also be administered prophylactically, for instance, before a subject manifests symptoms of infection with a fungus, to prevent or delay the development of infection with a fungus. Treatment may be performed before, during, or after the diagnosis or development of symptoms of infection. Treatment initiated after the development of symptoms may result in decreasing the severity of the symptoms of one of the conditions, or completely removing the symptoms.

Advantages of preventive or prophylactic therapy with peptides listed in Table 1 or a peptide of Formula I over currently available antifungal agents include lower potential for pre-existing or development of anti-fungal resistance which can lead to therapeutic failure and drug-related toxicity.

An ACP of the present invention may be introduced into mammals or birds at any stage of fungal infection.

A peptide of the present invention may also be administered as a therapeutic to treat a subject suffering from a bacterial infection. Therapeutic treatment is initiated after diagnosis of or the development of symptoms consistent with infection with a bacterium. Exemplary bacterial infections include, but are not limited to, an infection with Staphylococcus aureus, Escherichia coli, Streptococcus pneumoniae, Pseudomonas aeruginosa, Klebsiella pneumoniae, Salmonella senftenberg, Shigella sonnei, and Mycobacterium species, including M. tuberculosis and non-tuberculosis mycobacteria such as M. abscesses, M. chelonae, M. avium, and M. kansasii.

The ACPs of the present invention find utility in the treatment, control, or prevention of fungal or bacterial infection and disease not only in humans but also in animals. Compounds may be administered to companion animals, domesticated animals such as farm animals, animals used for research, animals in the wild, or birds. Companion animals include, but are not limited to, dogs, cats, hamsters, rabbits, gerbils, birds (including chickens, turkeys) and guinea pigs. Domesticated animals include, but are not limited to, cattle, horses, pigs, goats, sheep, and llamas. Research animals include, but are not limited to, mice, rats, rabbits, dogs, pigs, apes, and monkeys.

### Combination Therapy

The invention thus provides in a further aspect a combination comprising one or more peptides listed in Table 1, a peptide of Formula I, or a combination thereof together with one or more therapeutically active agents which, in one non-limiting embodiment, may be an antibiotic, antifungal, antiviral or other anti-infectives. As such, it will be appreciated that the pharmaceutical composition may further comprise at least one other pharmaceutically active agent, not necessarily an antimicrobial or anti-infective. Suitably, the pharmaceutically active agent may be selected from antibiotic agents, antibacterial, antifungal, and antiviral agents, or other anti-infectives. Non-limiting examples of therapeutic antifungal agents include polyenes, azoles allylamines, echinocandins, and others. Preferred examples of antifungal agents include amphotericin B, flucytosine, fluconazole, itraconazole, ketoconazole, miconazole, posaconazole, voriconazole, caspofungin, ibrexafungerp, micafungin and anidulafungin.

When combination therapy is employed with one or more compositions of the present invention, the additional therapy may be given prior to, at the same time as, and/or subsequent to the composition of the present invention.

### Kits

Any of the compositions described herein may be offered in a kit. In a non-limiting example, an antifungal composition of the present invention, such as one or more peptides listed in Table 1 or a peptide of Formula I, or a combination thereof may be combined in a kit. The kits may comprise a suitably aliquoted of a composition of the present invention and, in some cases, one or more additional agents, packaged either in aqueous media or in lyophilized form or as a solid dosage form in blister packs. The container means of the kits will generally include at least one vial, test tube, flask, bottle, syringe or other container means, into which a component may be placed, and preferably, suitably aliquoted. The kit may also contain instructions for use.

### EXAMPLES

### Example 1

Peptides are synthesized using standard solid phase peptide chemistry with FMOC protected amino acids on resin. Amino acid activation and couplings are carried out with HBTU/HOBt and DIEA, for example. FMOC groups are removed using 20% piperidine in DMF. After completion of the individual peptide syntheses, the resin-bound sequence was then cleaved from resin and deprotected with 80-90% trifluoroacetic acid (TFA) containing a variety of scavengers which can include water, thioanisole, ethylmethylsulfide, and ethanedithiol, and/or triisopropylsilane. Peptides are precipitated into ether and then isolated by centrifugation. The dried peptide pellets are reconstituted in a water and acetonitrile mixture and lyophilized prior to purification by reverse-phase HPLC on a C18 column, which is eluted with acetonitrile-water buffers containing 0.1% TFA. The peptide is analyzed and pure fractions are pooled and lyophilized. Analytical HPLC data is obtained on a 5-micron C18 analytical column and eluted with water-acetonitrile buffers containing 0.1% TFA. Molecular weight is confirmed by MALDI-TOF analysis. For salt conversion, anion exchange resin was used, either in the acetate or the chloride form. The purified peptide is dissolved in 20-50% acetonitrile in water, loaded on a strong anion exchange resin (desired salt form) and eluted with either 10% acetic acid in 30-50% acetonitrile in water for the acetate form, or just 30-50% acetonitrile for the chloride form. Results for ACPs are shown in Table 14.

TABLE 14
Molecular Weight and Purity of ACPs

| | HPLC Purity | |
|---|---|---|
| Seq ID NO. | (%) | Mol Wt. |
| 2 | 96.5 | 2479.0 |
| 3 | 100 | 2375.8 |
| 4 | 99.8 | 2374.8 |
| 5 | 97.7 | 2633.3 |
| 6 | 95.3 | 2633.3 |
| 7 | 100 | 2416.9 |
| 8 | 98.8 | 2417.9 |
| 9 | 100 | 2416.9 |
| 10 | 100 | 2416.9 |
| 11 | 99.4 | 2416.9 |
| 12 | 100 | 2416.9 |
| 13 | 99.3 | 2416.9 |
| 14 | 93.2 | 2416.9 |
| 15 | 98.0 | 2416.9 |
| 16 | 99.6 | 2559.3 |
| 17 | 97.0 | 1793.1 |
| 18 | 100 | 2500.6 |
| 19 | 99.7 | 2502.0 |
| 20 | 93.5 | 2631.1 |
| 21 | 100 | 2501.0 |
| 22 | 100 | 2188.9 |
| 23 | 100 | 1876.5 |
| 24 | 92.4 | 1876.3 |
| 25 | 98.7 | 1876.3 |
| 26 | 98.7 | 1876.3 |
| 27 | 100 | 1876.3 |
| 28 | 99.0 | 1876.3 |
| 29 | 92.7 | 2627.3 |
| 30 | 100 | 2628.2 |
| 31 | 99.2 | 2669.6 |
| 32 | 96.6 | 2656.6 |
| 33 | 98.1 | 2655.0 |
| 34 | 95.5 | 2798.0 |
| 35 | 94.3 | 2543.0 |
| 36 | 97.4 | 1918.3 |
| 37 | 97.7 | 2585.0 |
| 38 | 93.1 | 2543.3 |
| 39 | 92.2 | 1921.4 |
| 41 | 100 | 2502.1 |
| 42 | 96.8 | 2502.1 |
| 43 | 95.1 | 2502.1 |
| 46 | 100 | 2504.3 |
| 47 | 93.8 | 2503.0 |
| 48 | 100 | 2503.0 |
| 49 | 93.9 | 2503.0 |
| 51 | 90.8 | 2505.2 |
| 52 | 93.5 | 2507.0 |
| 53 | 100 | 2495.0 |
| 54 | 96.0 | 2496.0 |
| 55 | 100 | 2537.0 |
| 56 | 98.3 | 2573.5 |
| 57 | 100 | 2632.1 |
| 58 | 100 | 2604.2 |
| 59 | 99.6 | 2621.1 |
| 60 | 100 | 2495.0 |
| 61 | 99.5 | 2397.9 |
| 62 | 100 | 2300.8 |
| 63 | 100 | 2651.2 |
| 64 | 100 | 2807.4 |
| 65 | 99.0 | 2338.8 |
| 66 | 99.0 | 2182.6 |
| 67 | 97.4 | 2085.5 |
| 68 | 100 | 1870.3 |
| 69 | 91.7 | 1870.3 |
| 70 | 97.1 | 1870.3 |
| 71 | 100 | 1870.3 |
| 72 | 97.9 | 1870.3 |
| 73 | 100 | 1870.3 |
| 74 | 95.9 | 2537.0 |
| 75 | 100 | 2727.1 |
| 76 | 100 | 2681.4 |
| 77 | 100 | 2648.0 |
| 78 | 93.9 | 2023.2 |
| 88 | 90.1 | 2578.6 |
| 89 | 92.8 | 2584.7 |
| 93 | 97.7 | 2465.9 |
| 94 | 99.0 | 2465.9 |
| 95 | 98.6 | 2436.9 |

### Example 2

Arginine-containing peptides (ACPs) were tested for antifungal activities in panels of fungal strains using in vitro broth microdilution assay under the assay conditions described by the Clinical and Laboratory Standards Institute (CLSI). Yeast and fungi were tested in the medium RPMI-1640 buffered to pH 7.0 with 0.165 M 3-N-morpholinepropane sulfonic acid (MOPS). The Minimum Inhibitory Concentration (MIC) is defined as the lowest concentration of an agent that inhibits visible growth of the microorganism. Test articles were dissolved in phosphate buffered saline (PBS) and diluted by 2-fold serial dilutions in PBS for a total of 11 test concentrations. Deep-well polypropylene 96-well plates were used to first create 10× of the serially diluted test article concentration solutions, followed by 1:5 dilution into 125% the medium (RPMI-1640 with MOPS) to make the 2× of test concentration solutions. Then 100 μL of each of 2× test concentration solution was added to each well of another 96-well plate followed by adding 100 μL of the appropriate innocula prepared in the medium resulting in final concentrations of approximately 0.4 to 5×10³ colony forming unit (CFU)/mL (Candida spp., Cryptococcus spp., Coccidioides spp., and Rhizopus spp.), 0.4 to 5×10⁴ CFU/mL (Fusarium spp., Scedosporium spp., and Paecilomyces variotii) and 1.5×10³ CFU/mL (dermatophytes). The plates were incubated aerobically at 35° C. without agitation for 24 hrs (Candida spp. and Rhizopus spp.), 48 hrs (Fusasium spp. and P. variotii), 72 hrs (Cryptococcus spp. and Scedosporium spp.), 48-72 hrs (Coccidioides spp.) and 4-6 days for dermatophytes, with the MIC values reported at >50% inhibition for filamentous fungi, Coccidioides spp. and dermatophytes and complete (100%) inhibition for yeast. For reference compounds, MICs were read at >50% inhibition for azoles and echinocandins and 100% inhibition for amphotericin B following CLSI guidelines. Growth control wells contained 100 μL of fungal suspension and 100 μL of the growth medium without test article or positive control agent (amphoterin B, fluconazole, voriconazole, posaconazole and/or caspofungin). The ACPs were tested by batches at different times, each with 2-7 strains of C. albicans (including strains resistant to fluconazole and/or caspofungin), 2-8 strains of Candida glabrata (including strains resistant to fluconazole and/or caspofungin), 2-3 strains of Candida tropicalis (including strains resistant to fluconazole), 3-6 strains of Candida parapsilosis (including strains resistant to fluconazole), 2-3 isolates of Candida krusei (including strains resistant to fluconazole), 4-8 strains of Candida auris (including strains resistant to fluconazole), 1 strain of Candida dubliniensis, 2-5 strains of Cryptococcus neoformans (including strains resistant to fluconazole and/or caspofungin), 1 strain of Cryptococcus gattii (resistant to caspofungin), 3-6 strains of Fusarium spp. (including F. falciforme, F. oxysporum, and F. solani as well as strains resistant to voriconazole, fluconazole and/or caspofungin), 2-4 strains of Scedosporium spp. (including strains of S. boydii and S. apiospermum as well as strains resistant to fluconazole), 1 strain of Lomentospora prolificans (including strains resistant to voriconazole), 3-10 strains of Coccidioides spp. (including C. immitis and C. posadasii as well as strains resistant to fluconaozole), 1 strain of Paecilomyces variottii, 3 strains of Rhizopus arhizzus, and 1 strain each of Trichophyton rubrum, Epidermophyton floccosum, and Microsporum gypseum.

The MIC data in Tables 3 to 6 show that the ACPs possess potent antifungal activity as compared to the positive reference compounds in a broad spectrum of important fungal species including strains that are resistant to current therapies.

### Example 3

A time-kill kinetic study was executed as described by Canton et al. (Canton E et al. Antimicrob Agents Chemother 2007, 53(7): p. 3108-11) to evaluate the fungicidal activity of the ACPs. SEQ ID NO: 7, SEQ ID NO: 8 and SEQ ID NO: 32 were tested at 2× and 8× the MIC value determined by broth microdilution.

For each test, a well of a deep well 96-well assay plate (Costar 3960) contained 900 μL RPMI-1640, 100 μL fungal inoculum (1 to 5×10⁶ CFU/mL), and 2 μL test agent. A drug-free control well containing RPMI-1640, inoculum and 2 μL PBS served as growth controls for each isolate. After inoculation, the deep-well plates were incubated at 35° C. with shaking at 200 rpm. Viable yeast were quantified at timepoints 1, 2, 4, 6, and 24 hr post-inoculation for the Candida albicans ATCC strain 90028 and at 1, 2, 6, 24, and 72 hr for the Cryptococcus neoformans ATCC strain MYA-4564. At Time=0 hr, a 0.1 mL aliquot was removed from each inoculum suspension, 10-fold serial-diluted in chilled sterile PBS, and track dilution plated to determine the CFU/mL at 0 hr. During track dilution plating, a 10 μl aliquot of each dilution was spotted across the top of a square Sabouraud Dextrose Agar plate. The plate was then tilted at a 45-900 angle to allow the 10 μL aliquot to track across the agar surface. The plates were laid flat, dried at room temperature, then inverted and incubated at 35° C. for ˜24 hr for C. albicans or 48 h for C. neoformans. CFU/mL was then determined from the average colony count of duplicates with a limit of detection of 50 CFU/mL. A reduction of CFU of at least 3-logs from the starting inoculum is considered as fungicidal.

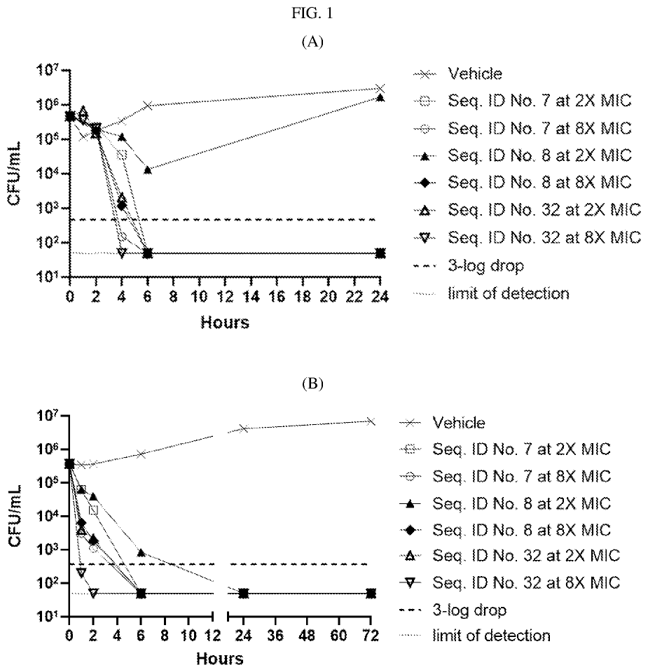

The results are shown in FIG. 1A for C. albicans and FIG. 1B for C. neoformans. In both cases, the three ACPs showed rapid and significant fungicidal activity with a ≥3-log drop in CFU/mL compared to time 0 h and this activity is comparable to or superior to the time-kill activity of approved antifungal agents for these species.

### Example 4

Arginine-containing peptides (ACPs) were tested for antibacterial activities in a panel of bacterial species see using the in vitro broth microdilution assay under assay conditions described by CLSI. Cation Adjusted Mueller Hinton broth (CAMHB) was used for MIC testing. The Minimum Inhibitory Concentration (MIC) is defined as the lowest concentration of an agent that completely inhibits visible growth of the microorganism. Test articles were dissolved in phosphate buffered saline (PBS) and diluted by 2-fold serial dilution in the same vehicle for a total of 11 test concentrations. A 4 μL aliquot of each dilution was added to 196 μL of broth medium seeded with the organism suspension in wells of a 96 well plate (final bacterial count: 2-8×10⁵ colony forming units/mL per well). Plates were incubated at 35 or 36° C. for approximately 16-24 hrs or 48 hrs (Mycobacteria spp.). Following incubation, the test plates were visually examined, and wells were scored for growth or complete growth inhibition to define the minimum inhibitory concentration. Vehicle-control and appropriate active reference agents were used as blank and positive controls, respectively.

The MIC values in Table 7 show that the ACPs possess antibacterial activity.

### Example 5

The ACPs did not cause any hemolysis of human red blood cells tested at concentrations up to 300 μg/mL, which is substantially higher than their antifungal or antibacterial MICs. Hemolysis is a liability for many other cationic peptides which prevented their utility to treat systemic infections. The hemolytic potential of the peptides was tested using red blood cells collected from fresh human blood after centrifugation at room temperature and washed in phosphate buffered saline (pH 7.4) three times and then incubated in phosphate buffered saline (PBS) at 37° C. for 1 hr with the peptides at concentrations of 3-300 μg/mL. Triton-X100 was used as the positive control while the vehicle (PBS) was used as the negative control. Amphotericin B and melittin, both known to be hemolytic were used as reference compounds. Following incubation, the mixture was centrifuged at room temperature, and the supernatant was separated and analyzed for light absorbance at a single wavelength of 410 nm. The background absorbance reading from the negative control was subtracted from all samples. The Triton-X-100 sample was used to represent 100% lysis. All test compound and positive control samples were normalized to this value to determine the percent lysis caused by the test compounds and the positive controls at each concentration. EC₅₀ value (the concentration of test article that produced a 50% lysis) was determined where possible for each test compound.

TABLE 15
No Hemolytic Potential of Arginine-containing
Peptides Incubated with Human Red Blood Cells

| | EC50 |
|---|---|
| SEQ ID NO. | (μg/mL) |
| 5 | >300 |
| 2 | >300 |
| 34 | >300 |
| 32 | >300 |
| 3 | >300 |
| 4 | >300 |
| 7 | >300 |
| 8 | >300 |
| 16 | >300 |
| 17 | >300 |
| 13 | >300 |
| 14 | >300 |
| 15 | >300 |
| 29 | >300 |
| 18 | >300 |
| 53 | >300 |
| 33 | >300 |

The results showed that the ACPs (Table 15) had no detectable hemolytic activity. In comparison, the two positive controls produced clear concentration related increases in hemolysis, with EC₅₀ of 2.63 μg/mL and 6.95 μg/mL for melittin and amphotericin B, respectively.

### Example 6

The ACPs have no or low potential of cytotoxicity in human hepatoma (HepG2) cells when tested at concentrations up to 300 μg/mL, which is substantially higher than their antifungal or antibacterial MICs. We tested for cytotoxicity of peptides using changes in ATP levels in HepG2 cells as an indicator for cell viability. Changes in the intracellular levels of ATP is indicative of cytotoxicity. ATP is the primary energy source of mammalian cells and tissues. Compounds that cause a reduction in cellular ATP have been shown to be cytotoxic. A human hepatoma cell line (HepG2) from American Type Culture Collection (ATCC, Cat #HB 8065) was used for assessing cytotoxicity. This cell line has been well characterized and has been used as a sentinel for chemical toxicity for many years. Healthy cells have high levels of ATP. If cells are stressed by drug exposure, ATP levels can decrease rapidly indicating a cytotoxic effect. ATP was monitored with CellTiter Glo® Luminescent Cell Viability Assay (Promega, Cat #G7572) for detecting ATP inside of cells. HepG2 cells were seeded into 96-well culture plates at a density of 20,000 cells per 100 μL. The cells were cultured in Eagles Minimum Essential Medium (EMEM) with 10% fetal bovine serum (FBS) at 37° C. and 5% CO₂. Following an equilibrium period of 18-22 hrs, the media (containing FBS) was removed, and cells were washed twice with media without FBS. Subsequently, 200 μL of media containing the peptides (at 1-300 μg/mL) without FBS or the positive controls melittin (at 0.01 to 10 μg/mL) and amphotericin B (at 1-100 μg/mL) without FBS was added. An Internal control was conducted to verify that the ATP assay was performing within historical values using rotenone (at 0.1 to 100 μM). The negative control was the vehicle, PBS plus EMEM media without FBS for the peptides and melittin, or DMSO (0.1%) plus EMEM media without FBS for amphotericin B and rotenone. A growth control of cells exposed to vehicle in complete EMEM (with FBS) was also done. Exposures to test and reference compounds were for 18-22 hrs at 37° C. with 5% CO₂. Following the exposure period, the media was removed, and 50 μL of fresh media plus 50 μL of lysis reagent (which contained luciferase) was added to cells and the plates were shaken for 10 min. The assay luminescence was read.

The raw data of relative luminescence units were obtained, and cell viability was calculated using the following equation. The mean data were converted into percent cell viability relative to vehicle control without FBS. The exposure concentration that resulted in a 50% viability (EC50) was estimated using GraphPad Prism 9 sigmoidal curve extrapolation with Hill Slope determination. The samples that caused cell viability to fall under 50% are considered cytotoxic in the concentration range tested.

$$\% Viability = \frac{sample luminescence}{average vehicle control luminescence} \times 1$$

TABLE 16
Lack of Cytotoxicity of Arginine-containing Peptides
in a Human Hepatoma (Liver) Cell Line
(HepG2) using an Intracellular ATP Assay

| | EC50 |
|---|---|
| SEQ ID NO. | (μg/mL) |
| 2 | 40 |
| 34 | 162 |
| 32 | >300 |
| 3 | >300 |
| 4 | >300 |
| 7 | >300 |
| 8 | >300 |
| 16 | >300 |
| 17 | >300 |
| 13 | >300 |
| 14 | >300 |
| 15 | >300 |
| 29 | >300 |
| 18 | >300 |
| 53 | >300 |
| 33 | >300 |
| 66 | >300 |
| 75 | 49.8 |
| 60 | >300 |
| 76 | 138.6 |
| 65 | >300 |
| 63 | >300 |
| 64 | >300 |
| 62 | >300 |
| 88 | >300 |

The results showed that all except four of the ACPs tested had no detectable cytotoxicity (Table 16), with EC₅₀'s that were all well above 300 μg/mL. In comparison, the positive controls produced clear concentration related increases in cytotoxicity, with EC₅₀ values of 2.01-3.07 μg/mL and 5.79-14.13 μg/mL for melittin and amphotericin B across studies, respectively. The EC₅₀ values for rotenone were 0.129-0.594 μM.

### Example 7

Acute toxicity and subacute toxicity of ACPs were tested in CD-1 mice. In the acute toxicity study, groups of CD-1 male or female mice were administered single intravenous or intraperitoneal escalating doses (n=2-3 per dose) of the peptides dissolved in normal saline for injection or in phosphate buffered saline. The intravenous dose was administered via tail vein with a slow push over 15-20 seconds. Doses were escalated based on tolerability. The animals were observed for 15 minutes after injection for acute signs of intolerance (e.g., mortality, convulsions, tremors, ataxia, sedation, etc.) and autonomic effects (e.g., diarrhea, salivation lacrimation, vasodilation, pilorection etc.). Subsequently, mice were observed at least twice daily for 24 hrs, or in some cases up to 48-96 hrs post injection for clinical signs and overall health, including body weight, ruffled/matted fur, hunched posture, edema, decreased alertness, hypothermia, salivation, irritation/wounds at injection site, inability to eat or drink, lethargy. Peptides (including SEQ ID NO: 32, SEQ ID NO: 7, SEQ ID NO: 8, SEQ ID NO: 16, SEQ ID NO:14, SEQ ID NO: 53, SEQ ID NO: 33) were tolerated by mice after a single intravenous dose up to 5 to 7.5 mg/kg, or after a single intraperitoneal dose up to 10-15 mg/kg.

In the subacute toxicity study, groups of CD-1 male or female mice were administered seven consecutive daily intraperitoneal doses (n=3 per dose) of the peptides (SEQ ID NO: 7, SEQ ID NO: 29, SEQ ID NO: 53) dissolved in normal saline for injection. Each peptide was evaluated at 2 to 3 dose levels ranging from 2.5 to 7.5 mg/kg/day. Mice were weighed at least once daily and observed at least twice daily for any abnormal findings and determination of overall health. At 24 hrs after the last dose, mice were humanely euthanized, with blood collected through cardiac puncture into K₂EDTA microtainers to assess hematological parameters. All three peptides were tolerated at the highest dose evaluated (7.5 mg/kg/day) for 7 days. No significant clinical or hematological adverse effects were observed, including no evidence of hemolysis.

### Example 8

Enzymatic digestion studies showed that an ACP, SEQ ID NO: 7, is resistant to trypsin digestion and that ˜50% remains intact after 6 hrs of incubation with an Arg-targeted endoproteinase. The peptide (267 μg/mL) was incubated with Trypsin (9 μg/mL) from porcine pancreas (Sigma Aldrich Cat T6567) at 37° C., or the peptide (427 μg/mL) was incubated with 3 μg/mL of Endoproteinase Arg-C(Sigma Aldrich Cat 11370529001) at 37° C. Samples were taken at 0.5, 1 and 6 hrs post incubation to determine peptide concentrations using an LC-MS/MS method. The percent peptide remaining at each time point up to 6 hrs after incubation with Trypsin was ˜100% of that without incubation (time 0), indicating no degradation. The percent peptide remaining at 6 hrs post incubation with Endoproteinase Arg-C was 50.5%, representing a degradation half-life of about 6 hrs. When the peptides SEQ ID NO: 7, SEQ ID NO: 8, SEQ ID NO: 13, SEQ ID NO: 15, SEQ ID NO: 29, SEQ ID NO: 53 (5 μM) were incubated with full strength of human serum at 37° C. for 2 hrs, almost 100% of peptides remained intact with all peptides except SEQ ID NO: 13 which had only about 40% remaining, indicating the importance of D-arginine replacement for enhancing serum stability.

The in vitro enzymatic and serum stability of the peptides is manifested by the in vivo plasma pharmacokinetic (PK) profile. A single 5 mg/kg intravenous injection (via tail vein) and a single 7.5 mg/kg intraperitoneal dose of SEQ ID NO: 7 were administered to 2 groups of CD-1 mice (n=3 per group). The peptide doses were well tolerated. Serial blood samples were collected from the saphenous vein into EDTA-K₂ as anti-coagulant at time points up to 4 hrs or 8 hrs after the intravenous or intraperitoneal doses. Plasma was separated and analyzed by an LC-MS/MS method as follows. Due to the small blood volume collected from each mouse, the plasma was pooled from 3 mice before analysis. A 50 μL of plasma was protein precipitated with a 100 μL solution of 300 ng/mL TAT peptide (GRKKRRQRRRPQ; SEQ ID NO: 99) as the internal standard in 5% trichloroacetic acid. After centrifugation, an aliquot of the supernatant was injected onto an HPLC column (Waters ACQUITY UPLC HSS T3, 2.1*50 mm, 1.8 μm) eluted with a gradient of mobile phase containing 0.1% perfluoropentanoic acid (PFPA) in water and 0.1% PFPA in acetonitrile. The peptide and the internal standard were detected using a Triple Quad 6500+ mass spectrometer operated with electro-spray ionization in the positive-ion SRM mode. The calibration curve range was from 10 to 4000 ng/mL. The mean plasma concentration of SEQ ID NO: 7 after the single intravenous and intraperitoneal doses are depicted in FIG. 2.

These results showed that the peptide achieved reasonably high plasma concentrations following a 5 mg/kg intravenous dose and had a reasonably long half-life of 1.43 hrs in mice. The plasma profile following an intraperitoneal injection showed that the peptide is substantially absorbed into the systemic circulation and survives first pass metabolism and pre-systemic degradation, with a bioavailability of approximately 75% (Table 17).

TABLE 17
Mean Pharmacokinetic Parameters of SEQ ID NO: 7 in Mice
Following a Single Intravenous (IV) or Intraperitoneal (IP) Dose

| | C₀ or Cₘₐₓ | tₘₐₓ | AUC | t_{1/2} | F |
|---|---|---|---|---|---|
| Dose Group | (μg/mL) | (hr) | (hr*μg/mL) | (hr) | (%) |
| IV | 4.48 | 0 | 4.42 | 1.43 | na |
| 5 mg/kg | | | | | |
| IP | 1.75 | 1.00 | 4.74 | 1.69 | 74.8 |
| 7.5 mg/kg | | | | | |

C₀ or Cₘₐₓ is concentration at time of injection for IV dose, or maximal concentration after IP dose; tₘₐₓ is the time when C₀ or Cₘₐₓ was observed; t₁/2 is plasma half-life; AUC is area under the plasma concentration vs. time curve; F is absolute bioavailability.

In another PK study, peptides SEQ ID NO: 29 and SEQ ID NO: 53 were administered to CD-1 mice intraperitoneally at 7.5 mg/kg once daily for 7 days. Plasma samples were collected from 3 mice at each time point for up to 6 hrs post dose on Day 1 and then from 3 mice at 6 hours post dose on Day 7. Samples were analyzed for concentrations of peptides using an HPLC-MS/MS as described above. Good in vivo plasma exposure similar to that presented above was also observed with these two peptides, along with substantial peptide penetration into the kidneys following daily 7.5 mg/kg intraperitoneal (ip) doses for 7 days in mice (Table 18). The Day 1 AUC ratios, kidney to plasma, were 158 and 58.3 for SEQ ID NO:29 and SEQ ID NO: 53, respectively.

TABLE 18
Kidney to Plasma Concentration Ratios in Mice

| Compound | Day 1 (n = 3) | Day 7 (n = 3) |
|---|---|---|
| SEQ ID NO: 29 | 236 ± 233 | 361 ± 322 |
| SEQ ID NO: 53 | 179 ± 106 | 916 ± 36 |

Mean ± SD at 6 hrs post dose of 7.5 mg/kg intraperitoneally.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the described embodiments, and without departing from the spirit and scope thereof, can make various changes and modifications of the embodiments to adapt it to various usages and conditions. Thus, other embodiments are also within the claims.

1. A peptide having the structure of Formula I:
S1-[block-1]ₘ-x-[block-2]ₙ-y-[block-3]ₒ-z-[block-4]ₚ-S2 Formula I
SEQ ID NO: 1
or a pharmaceutically acceptable salt thereof, wherein
m, n, o and p independently are 0 or 1, with 0 representing absent and 1 representing present, wherein at least two of m, n, o and p are 1;
block-1, block-2, block-3, and block-4 independently comprise 2 to 7 amino acids each independently selected from a L-arginine (R), D-arginine (r) and homoarginine (Har);
S1 and S2 are each independently an amino acid or amine acid other than an L-arginine (R), D-arginine (r) or homoarginine (Har), and are independently present or absent;
x, y, and z are each a linker, and each linker is, independently, present or absent and comprised of a single amino acid or amine acid selected from:
proline (P), glycine (G), 3-aminopropionic acid (β-alanine, Apr), 4-aminobutyric acid (Aba), 5-aminovaleric acid (Ava), 6-aminohexanoic acid (Ahx), 7-aminoheptanoic acid (Ahp), 8-aminooctanoic acid (Aoa), 9-aminononanoic acid (Ana), 10-aminodecanoic acid (Ada), 11-aminoundecanoic acid (Aun), 12-aminododecanoic acid (Ado), 13-aminotridecanoic acid (Atr), 14-aminotetradecanoic acid (Ata), 15-aminopentadecanoic acid (Apn), 16-aminohexadecanoic acid (Ahd), N-(3-aminopropyl)glycine (Apg), (S)-indoline-2-carboxylic acid (Ica), L-α-methyl-leucine (Leu(Me)), and L-2-indanylglycine (Igl), 5-amino-3-oxapentanoic acid (Aea), N-(2-aminoethyl)glycine (Aeg or Aeg2), isonipecotic acid (Inp), 2-cyclohexylglycine, N-butylglycine (ButylGly), N-(4-piperidinyl)glycine (PipGly), 2-amino-3-guanidino-propionic acid (Agp), (4′-pyridyl)alanine (4-PyrAla), (S)—N-(1-phenylethyl)glycine (Feg), N-benzylglycine (Bng), 1,2,3,4-tetrahydroisoquinoline-3-carboxylic acid (Tic), 1,2,3,4-tetrahydroisoquinoline-1-carboxylic acid (Tiq), and 4-guanidino-phenylalanine (Phe(4-Ngu));
with the provisos that when m is 1 and n is 0 or when n is 0 and n is 1, x is absent; when n is 1 and o is 0 or n is 0 and o is 1, y is absent; and when o is 1 and p is 0 or o is 0 and p is 1, z is absent;
optionally, the peptide has a modified N-terminal amino acid in which the N-terminal —NH₂ is replaced by —N(X₁)(X₂), wherein (X₁) and (X₂) are independently selected from H, R₁, R₂C(O), R₃SO₂, and R₄R₅NC(O), wherein R₁, R₂, and R₃ are independently an alkyl group or an alkaryl group, and R₄ and R₅ are independently H, an alkyl group, or an alkaryl group, and wherein the alkyl group and the alkaryl group, independently, are further optionally substituted with halogen, alkyl, amino, and/or oxygen moieties; and
optionally, the peptide has a modified C-terminal amino acid in which the C-terminal —COOH is replaced by —CONH₂ (carboxamide).

2. The peptide of claim 1, wherein S1 and S2 are absent.

3. The peptide of claim 1, wherein either S1 or S2 is absent.

4. The peptide of claim 1, wherein the peptide has a sequence selected from SEQ ID NOs: 2-98.

5. The peptide of claim 1, wherein at least two or three of m, n, o and p are 1, and block-1, block-2, block-3 and block-4 each has 2-4 amino acids independently selected from R, r and Har.

6. The peptide of claim 5, wherein all of m, n, o and p are 1.

7. The peptide of claim 1, wherein: m, n, o and p are all 1; block-1 and block-2 each has three amino acids; block-3 and block-4 each has four amino acids; x, y, and z are each Aoa; and each amino acid in block-1, block-2, block-3 and block-4 is independently selected from R, r and Har; optionally, the peptide has a modified C-terminal amino acid in which the C-terminal —COOH is replaced by —CONH₂.

8. The peptide of claim 7, wherein the peptide has the sequence of SEQ ID NO: 29.

9. The peptide of claim 1, wherein: m, n, o and p are all 1; block-1 and block-2 each has three amino acids; block-3 and block-4 each has four amino acids; x, y, and z are each P; and each amino acid in block-1, block-2, block-3 and block-4 is independently selected from R, r and Har; optionally, the peptide has a modified C-terminal amino acid in which the C-terminal —COOH is replaced by —CONH₂.

10. The peptide of claim 9, wherein the peptide has the sequence of SEQ ID NO: 53.

11. A peptide-conjugate, comprising the peptide of claim 1 and a group linked to the C-terminus or N-terminus, the group being selected from a polyethylene glycol (PEG) group, a glycosyl group, a lipid group, a cholesterol or sterol group, a peptide or protein group, and an oligonucleotide group.

12. A pharmaceutical composition, comprising the peptide of claim 1 and a pharmaceutically acceptable carrier, binder, diluent, or excipient.

13. A pharmaceutical composition, comprising a peptide-conjugate and a pharmaceutically acceptable carrier, binder, diluent, or excipient, wherein the peptide-conjugate includes the peptide of claim 1 and a group linked to the C-terminus or N-terminus, the group being selected from a polyethylene glycol (PEG) group, a glycosyl group, a lipid group, a cholesterol or sterol group, a peptide or protein group, and an oligonucleotide group.

14. A method of treating a microbial infection in a subject, comprising administering to a subject in need thereof a pharmaceutical composition containing the peptide of claim 1 and a pharmaceutically acceptable carrier, binder, diluent, or excipient.

15. The method of claim 14, wherein the microbial infection is a fungal infection.

16. The method of claim 15, wherein the fungal infection is an infection with a fungus selected from Absidia spp., Acremonium spp., Actinomadura spp., Apophysomyces spp., Arthrographis spp., Aspergillus spp., Basidiobolus spp., Beauveria spp., Blastomyces spp., Blastoschizomyces spp., Candida spp., Chrysosporium spp., Cladophialophora spp., Coccidioides spp., Conidiobolus spp., Cryptococcus spp., Cunninghamella spp., Emmonsia spp., Epidermophyton spp., Exophiala spp., Fonsecaea spp., Fusarium spp., Geotrichum spp., Graphium spp., Histoplasma spp., Lacazia spp., Leptosphaeria spp., Lomentaspora spp., Malassezia spp., Microsporum spp., Mucor spp., Neotestudina spp., Nocardia spp., Nocardiopsis spp., Paecilomyces spp., Paracoccidiomyces spp., Phialophora spp., Phoma spp., Piedraia spp., Pneumocystis spp., Pseudallescheria spp., Pyrenochaeta spp., Rhizomucor spp., Rhizopus spp., Rhodotorula spp., Saccharomyces spp., Scedosporium spp., Scopulariopsis spp., Sporobolomyces spp., Sporotrix spp., Syncephalastrum spp., Tinea spp., Trichoderma spp., Trichophyton spp., Trichosporon spp., Ulocladium spp., Ustilago spp., Verticillium spp., and Wangiella spp.

17. The method of claim 16, wherein the fungal infection is an infection with one or more of Candida spp., Coccidioides spp., Cryptococcus spp., Epidermophyton spp., Fusarium spp., Lomentospora spp., Microsporum spp., Paecilomyces spp., Rhizopus spp., Scedosporium spp., and Trichophyton spp.

18. The method of claim 14, wherein the microbial infection is a bacterial infection.

19. The method claim 18, wherein the bacterial infection is an infection with a gram-positive bacteria, gram-negative bacteria, or mycobacteria.

20. The method of claim 19, wherein the bacteria is Enterococcus faecium, Staphylococcus aureus, Escherichia coli, Klebsiella pneumoniae, Pseudomonas aeruginosa, Salmonella senftenberg, Shigella sonnei, or Mycobacterium spp.
